(19) United States
(12) Patent Application Publication
(10) Pub. No.: US 2025/0157662 A1
(43) Pub. Date: May 15, 2025

(54) PREDICTION MODELS FOR EARLY IDENTIFICATION OF PREGNANCY DISORDERS

(71) Applicant: DELFINA CARE INC., San Jose, CA (US)

(72) Inventors: Isabel Fulcher, Sunnyvale, CA (US); Ali Ebrahim, San Jose, CA (US); Senan Ebrahim, Rochester, MN (US); Noam Finkelstein, Sharon, VT (US); Jonathan Schor, San Francisco, CA (US); Adesh Kadambi, Mississauga (CA); Timothy Wen, San Francisco, CA (US)

(73) Assignee: DELFINA CARE INC., San Jose, CA (US)

(21) Appl. No.: 18/389,192

(22) Filed: Nov. 13, 2023

Related U.S. Application Data
(60) Provisional application No. 63/424,717, filed on Nov. 11, 2022.

(51) Int. Cl. G16H 50/30; G16H 50/20; G16H 50/70

(52) U.S. Cl. CPC G16H 50/30; G16H 50/20; G16H 50/70

(57) ABSTRACT

Embodiments include a computing device that executes software routines and/or one or more machine-learning architectures providing clinical predictive models to predict health complications resulting from pregnancy. The predictive models may identify and augment variables available in electronic health record systems during the first trimester of pregnancy and utilize machine learning methods to predict problematic outcomes later in the pregnancy or postpartum period. The prediction models follow several steps to identify possible pregnancy disorders, such as identifying data source and experts for classifier, collating data with clinical experts, applying statistical and machine learning methods, and assessing model performance and interpretability. The predictive models and related methods provide for unprecedented early detection of pregnancy complications for early intervention and treatment to improve health outcomes for the mother and child.

### CROSS-REFERENCE TO RELATED APPLICATION

This application claims priority to U.S. Provisional Application No. 63/424,717, filed Nov. 11, 2022, which is incorporated by reference in its entirety.

### TECHNICAL FIELD

This application generally relates to machine-learning architectures for healthcare, including prediction models for early identification of pregnancy disorders; and more specifically relates to machine learning models and techniques for predicting potential complications in the second and third trimesters of pregnancy.

### BACKGROUND

Disorders developed during pregnancy, such as hypertension, diabetes, and peripartum depression, can increase the risk of adverse maternal and neonatal outcomes. Hypertension in pregnancy complicates 10% of pregnancies and is the leading cause of pregnancy-related deaths in the United States. Gestational diabetes complicates about 4% of pregnancies and is associated with preterm delivery, macrosomia, and fetal malformation; in addition, 50% of women with gestational diabetes develop Type 2 diabetes after delivery. Early interventions, such as monitoring or initiation of medication or therapy, can reduce the risk of adverse outcomes and diagnoses. Thus, the ability to clinically identify patients at risk for a variety of pregnancy-related disorders in the first trimester is imperative to initiate interventions and improve pregnancy-related health outcomes.

Predefined clinical criteria, such as the United States Preventative Services Task Force (USPSTF) guidelines for preventive low-dose aspirin in persons at high risk for preeclampsia and the American Congress of Obstetricians and Gynecologists (ACOG) guidelines for early gestational diabetes screening in women with risk factors, currently guide clinical care to detect pregnancy complications. Both guidelines define “risk” as a simple rules-based algorithm, meaning that a person would be classified as “high risk” if they satisfy any one characteristic from a short list and “low risk” if they satisfy none. This approach is overly simplistic as it does not: consider the relative importance of each clinical characteristic, account for multiplicative effects when multiple conditions are met, consider risk as a continuous measure with uncertainty, and include additional clinical factors, such as vital signs or laboratory data, that are collected as part of standard prenatal care.

Incidences of Hypertensive Disorders of Pregnancy (HDP), for example, doubled in the United States from 2008 to 2019. Almost a quarter of maternal deaths that occurred during delivery hospitalization had a diagnosis code for HDP documented. Early identification of patients who would benefit from interventions may reduce incidence of HDP. Current ACOG risk criteria can also be improved upon by using machine learning.

### SUMMARY

Current technological approaches and standards associated with healthcare data present challenges to implementing machine-learning architectures for predicting pregnancy disorders. As an example, certain industry interoperability standards for generating, storing, hosting, and sharing healthcare data may present challenges in accessing certain types of data needed for a machine-learning architecture to make predictions of pregnancy disorders. Disclosed herein are systems and methods capable of addressing the above-described shortcomings and may also provide any number of additional or alternative benefits and advantages. Embodiments of the present disclosure relate to, among other things, a clinical curation process to identify and augment variables available in electronic health record systems during the first trimester of pregnancy and then utilize machine learning methods to predict problematic outcomes later in the pregnancy. In an exemplary embodiment, the process may follow four steps that may include identifying data source and experts for classifier, collating data with clinical experts, applying statistical and machine learning methods, and assessing model performance and interpretability.

In an embodiment, a computer-implemented method comprising: determining an identification of a pregnancy disorder outcome; receiving input data for training data, wherein the input data includes respective pluralities of health parameters for respective prior patients having experienced the pregnancy disorder outcome, selecting respective outcome-relevant sub-pluralities of the respective pluralities of health parameters, wherein the selecting is based upon a known outcome relevance or a computed outcome relevance based upon a machine learning output; allocating respective outcome-relevant sub-pluralities of parameters for respective patients into training data and testing data; fitting one or more machine learning models using the training data; evaluating the performance of the one or more machine learning models using the testing data to select a best performing machine learning model based upon statistical comparisons of performance among the one or more machine learning models; and updating, by the computer, a classification threshold for the selected machine learning model as a risk probability cutoff for a given patient developing the pregnancy disorder outcome.

In an embodiment, a computer-implemented method for predicting a pregnancy disorder outcome in a patient, comprising: collecting a plurality of health parameters for the patient prior to or within the first trimester of pregnancy; inputting the plurality of health parameters into a machine learning model; and determining, from an output of the trained machine learning model, a risk for the patient developing the pregnancy disorder during the pregnancy, wherein the machine learning model includes training by one or more training steps, comprising: receiving input data, wherein the input data includes respective pluralities of health parameters for respective prior patients having experienced the pregnancy disorder outcome, determining exclusion conditions for excluding any of the respective pluralities of health parameters based upon a determination of whether any respective prior patients meet the exclusion conditions; selecting respective outcome-relevant sub-pluralities of the respective pluralities of health parameters, wherein the selecting is based upon a known outcome relevance or a computed outcome relevance based upon a machine learning output; allocating respective outcome-relevant sub-pluralities of parameters for respective patients into training data and testing data; fitting one or more machine learning models using the training data; evaluating the performance of the one or more machine learning models using the testing data to select a best performing machine learning model based upon statistical comparisons of performance among the one or more machine learning models; and determining a classification threshold for the selected machine learning model as a risk probability cutoff for a given patient developing the pregnancy disorder outcome.

In an embodiment, a computer-implemented method for predicting a pregnancy disorder outcome in a patient, comprising: collecting, by a computer, a plurality of health parameters for the patient prior to or within the first trimester of pregnancy; obtaining, by the computer, the plurality of health parameters into a trained machine learning model having a performance for predicting risk of the outcome; and determining, by the computer, from an output of the trained machine learning model, a risk for the patient developing the pregnancy disorder during the pregnancy.

In an embodiment, a system for predicting a pregnancy disorder outcome in a patient, comprising a computing device. The computing device is operable to execute computer-readable instructions, the computer-readable instructions being configured to perform the steps of: receiving a plurality of health parameters for the patient prior to or within the first trimester of pregnancy; inputting the plurality of health parameters into a trained machine learning model having a performance for predicting risk of the outcome of at least about 0.7 as evaluated by area under the curve (AUC); determining, from an output of the trained machine learning model, a risk for the patient developing the pregnancy disorder during the pregnancy; and outputting the risk as a numerical or semantic classification value.

In an embodiment, a system for predicting a pregnancy disorder outcome in a patient, comprising a computing device. The computing device operable to execute computer-readable instructions, the computer-readable instructions being configured to perform the steps of: receiving a plurality of health parameters for the patient prior to or within the first trimester of pregnancy; inputting the plurality of health parameters into a machine learning model; determining, from an output of the trained machine learning model, a risk for the patient developing the pregnancy disorder during the pregnancy; and outputting the risk as a numerical or semantic classification value, wherein the machine learning model includes training by one or more training steps, comprising: receiving input data, wherein the input data includes respective pluralities of health parameters for respective prior patients having experienced the pregnancy disorder outcome, determining exclusion conditions for excluding any of the respective pluralities of health parameters based upon a determination of whether any respective prior patients meet the exclusion conditions; selecting respective outcome-relevant sub-pluralities of the respective pluralities of health parameters, wherein the selecting is based upon a known outcome relevance or a computed outcome relevance based upon a machine learning output; allocating respective outcome-relevant sub-pluralities of parameters for respective patients into training data and testing data; fitting one or more machine learning models using the training data; evaluating the performance of the one or more machine learning models using the testing data to select a best performing machine learning model based upon statistical comparisons of performance among the one or more machine learning models; and determining a classification threshold for the selected machine learning model as a risk probability cutoff for a given patient developing the pregnancy disorder outcome.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 shows components of a system according to an example embodiment;

FIG. 2 shows components of a system according to an example embodiment;

FIG. 3 is a chart depicting an exemplary timeline for early prediction and intervention of hypertension disorders of pregnancy;

FIG. 4 is a chart depicting the performance of an exemplary HDP model on internal validation cohort from a large academic medical center in the midwestern United States;

FIG. 5 is a chart depicting the performance of the exemplary HDP model in an external cohort of nulliparous pregnancies indicating that in external validation the model outperforms the ACOG checklist;

FIG. 6 is a chart depicting HDP model performance across different races/ethnicities;

FIG. 7 is a chart depicting an exemplary timeline for early prediction and intervention of GDM;

FIG. 8 is a chart depicting the performance of an exemplary model for detecting GDM;

FIG. 9 is a chart depicting an exemplary timeline for early prediction and intervention of excessive gestational weight gain (eGWG);

FIG. 10 is a chart depicting the performance of an exemplary excessive gestational weight gain model;

FIG. 11 is a chart depicting operational steps of an example process implementing predictive machine learning method;

FIG. 12 is a chart depicting the performance of an exemplary model for HDP in the first trimester compared with a reduced model;

FIG. 13 is a chart depicting the reduced model of FIG. 12 compared with USPSTF guidelines for aspirin initiation; and

FIG. 14 is a chart depicting the performances of exemplary gestational diabetes models in the first trimester.

### DETAILED DESCRIPTION

Reference will now be made to the illustrative embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to a person skilled in the relevant art and having possession of this disclosure, are to be considered within the scope of the invention.

Embodiments disclosed herein generally provide for early detection of pregnancy disorders, in many cases as early as the first trimester. Early detection and intervention are key in order to provide the mother and child with optimal health outcomes despite pregnancy disorder risks. For example, many traditional guidelines provide for pregnancy disorder detection no earlier than the third trimester or even after delivery of the child. Additionally, traditional guidelines are based upon limited insights which fail to capture additive or multiplicative effects of patient health parameters (such as characteristics or health status, demographics, lab results, etc.). The present invention in embodiments provides for methods for training machine learning models for much-needed early detection of pregnancy disorders, methods and systems for early detection of pregnancy disorders, and methods of treatment after early detection of a pregnancy disorder risk.

Embodiments described herein include computing systems comprising hardware and software components implementing machine-learning architectures for predicting health-related complications in pregnancies. A computing device executes software programming for the machine-learning architecture that includes classifiers trained to predict potential complications in the second and third trimester of pregnancy from health data routinely collected during the first trimester of pregnancy, such as biometric measures, laboratory values, and prior health history, which may indicate, and used to predict, risks of potential complications later in pregnancy. In some implementations, the computing device may implement a unique clinical curation process to identify and augment variables available in electronic health record (EHR) systems during a first time interval or trimester (e.g., 13 weeks) of the pregnancy and then applies the machine-learning architecture to predict problematic outcomes later in the pregnancy.

A machine-learning architecture includes machine-learning models or techniques trained for early prediction of patient health outcomes. The machine learning models may include, or include characteristics of, multi-layer perception (MLP) neural network, logistic regression (LR), ensemble (histogram gradient boosting, AdaBoost, LR, MLP), histogram-based gradient boosting, adaptive boosting (AdaBoost), histogram-based gradient boosting (bagged), random forest, generalized additive model (GAM), logistic regression+LASSO, and stochastic gradient boosting. These are non-limiting examples of machine learning models and various models may be implemented as described herein to produce predictive models for early detection of pregnancy disorders.

The machine-learning models of the machine-learning architecture may include one or more classifiers trained with training data comprising various types of patient care data. The training data typically includes various health parameters for prior patients having a diagnosis with a pregnancy disorder. In an embodiment, the data sources are identified by a quantitative or iterative process. In further embodiments, clinical experts are involved in the selection of data sources based upon several factors including, but not limited to: the feasibility of data acquisition, the source population, and containment of relevant data types (see, e.g., Table 1). Where necessary, clinical experts may guide the selection of appropriate data sources.

TABLE 1
Data types (i.e. health parameter types) with descriptions and examples

| Data type | Description |
|---|---|
| Clinical data | Information collected during pre-pregnancy care visits, at the first prenatal care |
| | visit, or at subsequent pregnancy-related visits (prenatal, labor and delivery, |
| | postpartum, and emergency). The following information is typically available in |
| | the electronic health record for the pregnant person and their infant(s) in discrete |
| | fields, structured/unstructured text fields, or as diagnostic codes |
| | (e.g. International Classification of Diseases (ICD) or Current Procedural |
| | Terminology (CPT) codes): |
| | Maternal pre-existing conditions, such as chronic hypertension or |
| | asthma |
| | Current and past medications prescribed to the pregnant person |
| | Past obstetric history, such as the number of prior pregnancies or the |
| | occurrence of gestational diabetes in a past pregnancy |
| | Maternal family's health history, such as diagnosis with hypertension or |
| | diabetes |
| | Vitals, such as blood pressure, heart rate, glucose levels, and weight, |
| | which may be collected at each prenatal visit or through remote patient |
| | monitoring |
| | Diagnoses for pregnant person, such as a maternal diagnosis with |
| | gestational diabetes, or infant, such as neonatal diagnosis with |
| | hypoglycemia |
| Laboratory | Results from standard laboratory and screening tests that occur during |
| and screening | pregnancy: |
| test results | Sexually transmitted infections (STI) - positive, negative, or |
| | indeterminate determination for a specific STI (e.g., chlamydia, |
| | gonorrhea, syphilis) |
| | Complete blood count - numeric values of hematocrit, hemoglobin, |
| | PAPP-A, and white blood cell count |
| | Blood type and Rh factor |
| | Urinalysis or urine culture - hCG value, presence of bacteria indicating |
| | urinary tract infection, proteinuria |
| | Ultrasound - gestational age dating in weeks and days, numeric value |
| | for nuchal translucency, and detection of structural abnormalities |
| | Fetal heart monitoring (nonstress test) - entire fetal heart rate trace |
| | Glucose tolerance tests - blood glucose level |
| Behavioral | Patient-reported current and historical tobacco, alcohol, and drug use. Some data |
| factors | systems may collect additional information on exercise and diet. |
| Socio- | Patient's age, sex, gender, race, ethnicity, and insurance type. Some data systems |
| demographic | may also collect information on a patient's education history, marital status, |
| factors | employment status, housing status, household income, experiences of intimate |
| | partner violence, experiences of obstetric racism, access to the internet, and |
| | adequacy of social support. |
| Population- | Socioeconomic or environmental factors that have been aggregated to a |
| level data | geographic level (e.g., census tract or postal code) and then matched to the |
| | patient depending on their place of current or prior residence or their clinic's |
| | location. These often come from publicly available data sources, such as the: |
| | Social Vulnerability Index, Environmental Justice Index, Food Access Research |
| | Atlas, County Health Rankings, PLACES Project, and the Area Deprivation |
| | Index. |

In various embodiments, data from selected data sources may be collated by an automated process or processes including input from clinical experts. In many cases, the pregnancy disorder outcome (i.e., the health outcome) may need to be defined. For example, a health outcome may already exist as a discrete field in a dataset or it may need to be developed based on multiple inputs. If the output needs to be developed based on multiple inputs, various factors may be considered, including, but not limited to, clinical criteria based on laboratory and screening test results, and combination of diagnostic codes. In embodiments, for clinical criteria based on laboratory and screening test results, to determine if a prior patient has been diagnosed with a condition, this may rely on various measurements recorded in the health record. For example, to determine if someone has developed preeclampsia, they must have blood pressure over 140/90 and evidence of kidney problems. For development using diagnostic codes, multiple diagnostic codes can be combined to represent a certain health outcome. For example, to determine if someone has developed preeclampsia, diagnostic code O14.10 refers to “severe preeclampsia in an unspecified trimester” and diagnostic code O14.05 refers to “mild to moderate preeclampsia in the third trimester”. In this case, experts would determine that anyone who has a code that starts with “O14” has been diagnosed with preeclampsia.

In various embodiments, health parameters may be selected by availability, quality, and relevance. For example, see, e.g. Table 1 for exemplary health parameters. In embodiments, the model may be flexible to include other types of health parameters as needed by the particular implementation. Parameters may be assessed for inclusion in models based upon several factors, including, but not limited to: availability in standard electronic health records, evidence that the parameter may be relevant to the outcome, and inclusion of socioeconomic parameters. Additionally, relevance between parameter and the outcome may be discovered by computation, for example, by discovery in machine learning training, testing, or implementation. Therefore, in some embodiments, the included parameters may be reassessed iteratively in the model development process.

In various embodiments, although the parameter may be available in the selected data source(s), it may be advantageous to determine if the parameter will be readily available for the majority of patients (about 80% complete or greater) in other health data systems where the classifier will be applied. For example, the receipt of a Complete Blood Count blood test is part of routine antenatal care, and the results of this blood test should be available in health data systems. In addition to data availability, the quality of the parameter in standard health records may also be considered, where “data quality” is an assessment of whether the parameter is fit to inform clinical decisions and involves evaluating accuracy and standardization of a given parameter. Accuracy means that the value of the parameter reflects the truth; standardization means the parameter values will be standardized across clinical practices.

In various embodiments, evidence (clinical or computed) may be available indicating that experts may select parameters that may be associated with the outcome based on their clinical knowledge and consensus of the scientific community. For example, prior diagnosis with anxiety would be included in a model for gestational diabetes due to the wealth of literature detailing the link between anxiety and gestational diabetes. Additionally, socioeconomic parameters may be included. In embodiments, to avoid propagating systematic biases into a model, race and ethnicity, income, and other socioeconomic parameters may be included in model development if they are deemed clinically relevant to the outcome.

In some embodiments, health records for a particular patient may not be perfect for a variety of reasons. In embodiments, the methods and systems herein may handle missing values in health records to maintain robust models. In some cases, the reason behind missing values may be determined. For example, individuals with missing screening tests may actually be less likely to have that condition, meaning that a missing value for a parameter is meaningful. Various other missing parameters may be meaningful, while other missing parameters may be missing due to incomplete records or non-meaningful reasons. The meaningfulness may be determined by knowledge or investigation, or may be determined computationally by iterative machine learning training processes as described herein.

In various embodiments, the machine learning models will be trained under, and will be useful in, certain patient populations, i.e., patients who do not meet exclusion criteria or inclusion criteria. For example, a specific population (based on clinical or sociodemographic factors) for which the classifier is relevant may be determined. For example, only individuals without pregestational diabetes can be diagnosed with gestational diabetes. As such, individuals with pregestational diabetes will be excluded from the development and testing of a classifier for gestational diabetes. Stated differently, pregestational diabetes may be an exclusion criterion for gestational diabetes, as a non-limiting example.

Various data handling steps may be performed upon the input data and health parameter data as necessary for a given implementation. For example, selected parameters may be combined for the chosen study population at the individual level. In an embodiment, additional pre-processing of parameters may be performed before model fitting, including transformation of existing parameters, dealing with missing values, and featurization of complex inputs.

In an embodiment, health parameter input data may be transformed. Said transformation may include combining existing parameters into one parameter. For example, height and weight measurements are combined into a body mass index. In addition, it may be advantageous to categorize continuous parameters based on clinically relevant cutoffs. For example, a continuous measure of body mass index may be categorized as “underweight”, “healthy weight”, “overweight”, or “obese”.

In various embodiments, missing values may be handled. For example, if the missing value itself is deemed informative, then the missing value may be encoded as a missing category, allowing the model to learn from the missing values. If the missing value itself is not deemed informative, then the value is imputed using an imputation approach, such as K-nearest neighbors. It can be appreciated that other methods for handling missing variables may be implemented as necessary.

In various embodiments, complex health parameter inputs may be handled. For example, a health parameter may include a complex signal of a dependent variable (health parameter) measured over some independent variable such as time. In an embodiment, featurization provides a method of transforming varied forms of data to numerical data for use in the machine learning models. For example, for continuous time series data of fetal heart rate, featurization involves extracting a finite set of measurements related to time and frequency.

Training of machine learning models generally relies upon utilization of a training data set as well as a testing set. In an embodiment, individuals are randomly allocated to a “training set” or a holdout “testing set”. The training set may be used to construct the classifier and the testing set may be used to determine the performance of the classifier. In some cases, a categorical outcome parameter may have an imbalanced distribution. To prevent skewing in favor of the majority, balancing may be performed upon the training data set. Examples of balancing methods include balanced class weights, undersampling the majority class, or oversampling the minority class. It should be appreciated that any balancing techniques are contemplated so as to improve the reliability of machine learning models.

In an embodiment, various machine learning models are fit in order to identify high-performing, or the best-performing, models. Examples of machine learning models include logistic regression, random forest or any other known models. Logistic regression estimates parameter values (i.e., log odds ratios) for each parameter included in the model, which can be used to return a probability of a binary outcome (e.g., gestational diabetes diagnosis) for each individual based on their variable values. A random forest model uses random subsets of training data, sampled with replacement, to generate a collection of decision trees to predict the outcome. The overall outcome of the random forest is selected by majority consensus among all trees, allowing for strengths of one tree to compensate for weaknesses or gaps in others. In various embodiments, the methods include specification of hyperparameter(s). For example, random forests require the number of decision trees to be specified. The value of the hyperparameters are determined by further splitting the training data into K datasets (where K is any positive integer), enumerating possible values for the hyperparameter(s), and then testing the model performance under all values of the hyperparameter(s). The hyperparameter(s) that have the highest performance are typically chosen.

In an embodiment, to improve model interpretability (i.e., for ease of explanation to clinicians), variable selection methods may be utilized to create a “reduced model” with a subset of variables. For logistic regression models, least absolute shrinkage and selection (LASSO) may be employed, which shrink the relative contributions of each variable towards zero depending on their importance. For other models, recursive feature elimination (RFE) may be employed. RFE iteratively removes variables by fitting the given machine learning algorithm, ranking features by importance, discarding the least important features, and re-fitting the model. This process may be repeated until the number of features remains that balances model interpretability (e.g., fewer variables) and model performance.

The model performance and interpretability are generally assessed during the optimization process. For example, certain performance metrics may be relied upon to assess the performance of a given model and to compare the relative performances of various models. In an embodiment, evaluation metrics include plots of the receiver operator characteristic (ROC) curves and the area under the curve (AUC). The ROC curve is threshold-agnostic, meaning that the performance of the model is displayed without a specific sensitivity or specificity threshold in mind-allowing one to choose the desired cutoffs based on a clinical objective. AUC is often used as the primary performance metric for prediction models as it provides a numeric summary for the ROC curve. The 95% confidence intervals (CI) for each AUC may be calculated using the fast DeLong method. In various embodiments, the “best” model may be chosen based on statistical comparison of the AUC between models using DeLong's test. If a model has a significantly (p-value<0.05) higher AUC than all other models, this model may be chosen for clinical use. However, if there are any ties between model performance, the most clinically interpretable model may be selected. In various embodiments, the models herein may achieve an AUC value of at least about 0.7 or higher.

In various embodiments, if applicable for a given outcome, the performance of the prediction model may be compared with existing classifiers to describe how the model outperforms current paradigms of clinical care. For example, the USPSTF recommends aspirin at 12 weeks of gestation in patients who are at high risk of preeclampsia where “high risk” is defined as any individual with preeclampsia in past pregnancy, carrying more than one fetus, chronic hypertension, kidney disease, diabetes mellitus, autoimmune conditions, or having multiple moderate risk factors. In various embodiments, after the “best” model has been determined, a probability cutoff for classifying an individual as “high risk” for the given outcome is determined. Therefore, in some embodiments, a predictive outcome may include a numerical probability, or may include a semantic risk classification of “high risk”, “moderate risk”, “average risk”, etc. In various embodiments, numerical or semantic risk outcomes may be utilized to inform treatment or intervention of the disease for which the patient is at risk.

The pregnancy disorder outcomes as described herein may include various disorders such as gestational diabetes, hypertensive disorders of pregnancy, excessive gestational weight gain, small for gestational age (i.e., small child for gestational age), and peripartum depression, among others. It should be appreciated that each outcome may be defined differently under different guidelines, and the model training process may take into account guidelines under which patients were diagnosed in the training data.

In an embodiment, a computing device may be used to perform computer-readable instructions. Such computer-readable instructions may be programmed to receive health parameters for the patient, input the patient health parameters into a trained machine learning model, determine a risk for the patient based on said input, and output the risk to the patient. It should be appreciated that the patient health parameters and risks to the patient may be displayed graphically through a patient portal or similar computerized display. This organized display of data may allow for better patient engagement and understanding.

### Components of Example Systems

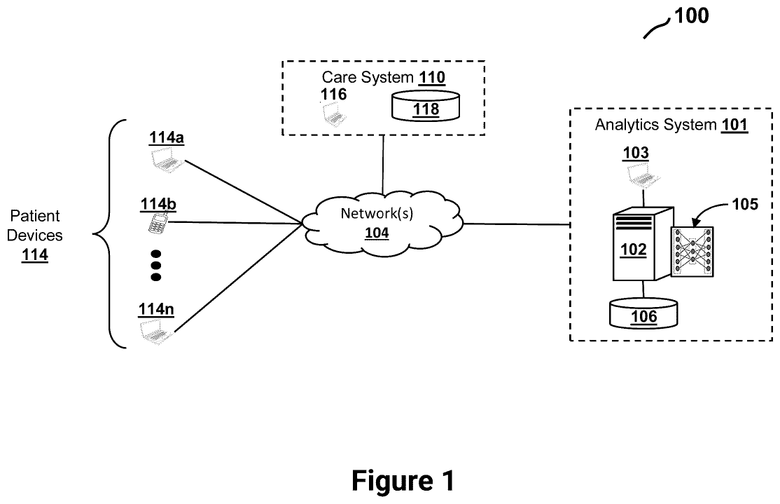

FIG. 1 shows components of a system 100 for predicting and caring for pregnancy disorders, according to an embodiment. The system 100 includes an analytics system 101, care systems 110, and patient devices 114a-114n (generally referred to as patient devices 114 or a patient device 114). The components of the system 100 may communicate with one another via one or more networks 104. The analytics system 101 and care systems 110 represent computing network infrastructures 101, 110 comprising physically and logically related software and electronic devices managed or operated by various enterprise organizations, including a healthcare analytics service and healthcare providers or similar organization (e.g., hospitals, clinics, physician office, insurance providers, research institutions). The analytics system 101 includes analytics servers 102 that execute software programming of a pregnancy prediction engine 105, admin devices 103, and analytics databases 106. The care system 110 includes provider devices 116 and provider databases 118. The system 100 depicted in FIG. 1 is merely an example. Embodiments may comprise additional or alternative components or omit certain components from those of FIG. 1, and still fall within the scope of this disclosure. Embodiments may include or otherwise implement any number of devices capable of performing the various features and tasks described herein.

The networks 104 host and conduct communications within the system 100. The networks 104 include various hardware components (e.g., switches, routers) and software components of one or more public or private networks, interconnecting the various components of the system 100. Non-limiting examples of such networks 104 may include: Local Area Network (LAN), Wireless Local Area Network (WLAN), Metropolitan Area Network (MAN), Wide Area Network (WAN), and the Internet. The communication over the networks 104 may be performed in accordance with various communication protocols, such as Transmission Control Protocol and Internet Protocol (TCP/IP), User Datagram Protocol (UDP), and IEEE communication protocols, implemented by the components of the networks 104, patient devices 114, and devices of the analytics system 101 and care systems 110.

In some embodiments, the system 100 includes the patient devices 114. The patient devices 114 may include any electronic computing devices comprising hardware and software components capable of performing the various processes and tasks described herein. Patients use the patient devices 114 to access and interact with a care application, which performs various operations that provide the patients outputs of the analytics servers 102, such as outputs of the pregnancy prediction engine 105. The patient device 114 may include an electronic device comprising a processor and/or software data streaming via a TCP/IP network, or other computing network channel. In some cases, the care application is natively installed on and executed by the patient device patient device 114, where the care application may contact the analytics server 102 in order to access and interact with certain features of the care application and related data. In some cases, the patient device 114 executes a web browser that accesses the features and functions of the care application, as hosted and executed by webserver software of the analytics server 102. Non-limiting examples of the patient devices 114 may include patient computers 114a, a Virtual Private Cloud (VPC), or patient mobile devices 114b, among other types of electronic computing devices. The patient computer 114a may include any type of computing device, such as workstation computers, laptops, tablets, servers, and the like. The patient mobile device 114b may include any type of mobile electronic computing devices, such as smartphones, tablets, or edge devices, among others.

The analytics system 101 hosts the care application, pregnancy prediction engine 105, and/or other computing services for gathering healthcare data to develop and execute software-based components of the pregnancy prediction engine 105. In some implementations, the care application of the analytics server 102 interacts with, and provides outputs to, the patient devices 114. Additionally or alternatively, in some implementations, the care application of the analytics server 102 interacts with, and provides outputs to, the provider devices 116.

The analytics server 102 includes any computing device comprising hardware (e.g., processors, non-transitory machine-readable storage memory) and software components (e.g., pregnancy prediction engine 105, webserver software) capable of performing the various processes and tasks described herein. Although FIG. 1 shows only a single analytics server 102, the analytics server 102 may include any number of computing devices. In some cases, the computing devices of the analytics server 102 may perform all or portions of the processes and benefits of the analytics server 102. The analytics server 102 may comprise computing devices operating in a distributed or cloud computing configuration and/or in a virtual machine configuration. It should also be appreciated that, in some embodiments, functions of the analytics server 102 may be partly or entirely performed by the computing devices of the care systems 110 or the patient devices 114.

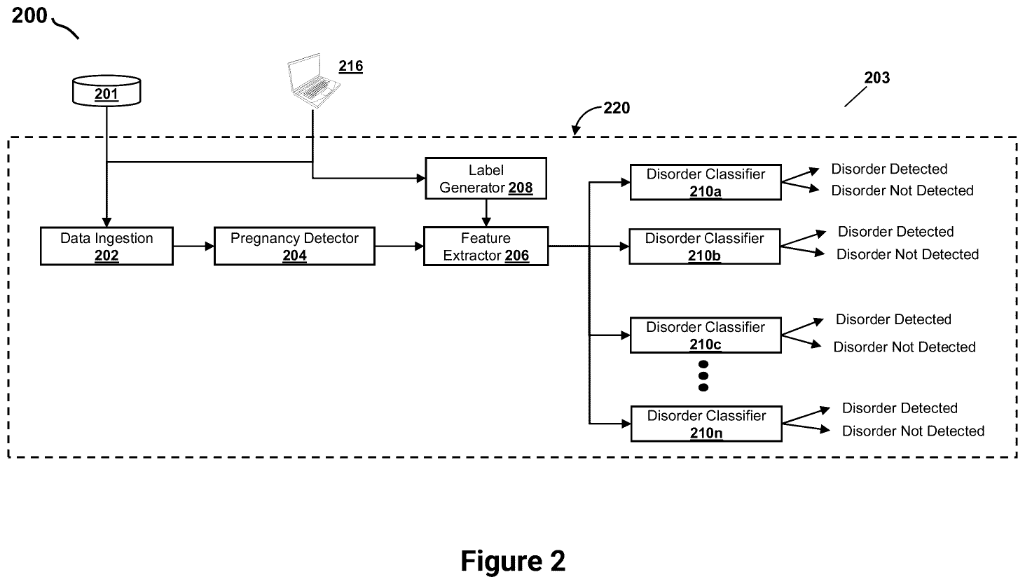

The pregnancy prediction engine 105 includes software programming for predicting and detecting one or more types of pregnancy disorders. As discussed further in FIG. 2, the pregnancy prediction engine 105 may include software routines defining certain operational engines and machine-learning models of a machine-learning architecture, among other potential operations for conditioning patient care data. The pregnancy prediction engine 105 takes as input patient care data, detects an instance of pregnancy, extracts features and feature vectors from the data available during the pregnancy instance, and applies one or more classifier models on the extracted feature vectors to determine disorder prediction scores indicating likelihoods of pregnancy disorders. The pregnancy prediction engine 105 detects a pregnancy disorder when the corresponding classifier trained to detect the given disorder determines that the disorder prediction score satisfies a detecting threshold.

The pregnancy prediction engine 105 operates logically in several operational phases, including a training phase and a deployment phase (sometimes referred to as “inference time”). At training time, the pregnancy prediction engine 105 extracts training features and training vectors from a training data set to generate various predicted outputs. The pregnancy prediction engine 105 or the users may compare the predicted outputs against labels containing expected outputs to determine a level of error. The users or machine-learning functions of the pregnancy prediction engine 105 may adjust or tune, for example, the algorithms, heuristics, data input types, hyperparameters, weights, thresholds, or other aspects of the functional engines of the pregnancy prediction engine 105 in order to reduce the level of error. At deployment time, the patient device 114 or the provider device 116 feeds patient data into the pregnancy prediction engine 105. The pregnancy prediction engine 105 extracts the patient's features and feature vectors from the patient care data to generate the various detection outputs.

The admin device 103 may include any computing device comprising hardware (e.g., processors, non-transitory machine-readable storage memory) and software components capable of performing the various processes and tasks described herein. Non-limiting examples of the admin device 103 may include a personal computer (e.g., workstation computer, laptop computer), mobile device, tablet, or the like. The admin device 103 includes a user interface allowing a system administrator user to interact with the configurations of the system 100, including the configurations of the pregnancy prediction engine 105. The administrator may enter various configuration inputs that, for example, adjust or tune, for example, the algorithms, heuristics, data input types, weights, and thresholds, among other aspects of the functional engines of the pregnancy prediction engine 105.

The analytics databases 106 may be hosted on any number of computing devices comprising hardware (e.g., processors, non-transitory machine-readable storage memory) and software components capable of performing the various processes and tasks described herein. The analytics database 106 may store patient care data for patients associated with the analytics system 101 (e.g., patients treated by clinicians who utilize the pregnancy prediction engine 105; patients who operate a patient device 114 that executes software utilizing the pregnancy prediction engine 105). The analytics database 106 may store configurations of the pregnancy prediction engine 105, as received from the provider devices 116 or admin devices 103, and/or as automatically tuned by the machine-learning models and functions of the pregnancy prediction engine 105. In some embodiments, the analytics database 106 may further contain various instances or versions of the pregnancy prediction engine 105, which may be tailored by the administrators or clinicians with configurations of different care systems 110 or patients.

As mentioned, the care systems 110 include computing network infrastructures for various clinical entities, such as hospitals, physicians' offices, clinics, or research organizations, among others. The hardware and software components of the care system 110 generate and host patient care data and may access and interact with the software services (e.g., care software, pregnancy prediction engine 105) hosted by the analytics system 101.

The provider devices 116 include any computing device comprising hardware (e.g., processors, non-transitory machine-readable storage memory) and software components capable of performing the various processes and tasks described herein. Non-limiting examples of the provider device 116 may include a personal computer (e.g., workstation computer, laptop computer), mobile device, tablet, or the like. In some cases, the provider device 116 includes a user interface allowing a clinician user (e.g., physician, healthcare worker, researcher) to interact with certain configurations of the pregnancy prediction engine 105 and/or the patient care data in the provider database 118 or analytics database 106. The administrator may enter various configuration inputs that, for example, adjust or tune, for example, the algorithms, heuristics, data input types, weights, and thresholds, among other aspects of the functional engines of the pregnancy prediction engine 105. The provider device 116 may, for example, enter certain configurations that indicate which types of data should be used for identifying a pregnancy instance or detecting a particular type of pregnancy disorder. In some cases, the provider device 116 contains patient care data in memory, and may provide the patient care data to the analytics system 101 as additional input patient care data inputs. The outputs generated by the analytics server 102 may be transmitted and presented to a clinician user interface of the provider device 116, so that the clinician may review the results with the patient or other processes.

The provider database 118 may be hosted on any number of computing devices comprising hardware (e.g., processors, non-transitory machine-readable storage memory) and software components capable of performing the various processes and tasks described herein. In some cases, the provider database 118 may include various types of electronic healthcare records (EHRs) of patients according to EHR standards, but embodiments are not so limited. The provider database 118 may include other types of data associated with patient care, other than formal or standardized EHR patient care data. The analytics server 102 may receive or retrieve the patient care data from the provider database 118, or the provider device 116 may transmit the patient care data from the provider database 118 to the analytics server 102.

FIG. 2 shows data flow amongst components of a system 200 for predicting and caring for pregnancy disorders, according to an embodiment. The system 200 includes data sources 201, user devices 216, and an analytics server 203. The analytics server 203 includes machine-executable pregnancy prediction software programming comprising software routines of a pregnancy prediction engine 220. The software components of the pregnancy prediction engine 220 may include functions for certain operational engines that define a machine-learning architecture. As shown in FIG. 2, the pregnancy prediction engine 220 includes, for example, software programming that define, or perform functions of, a data ingestion engine 202, pregnancy detector 204, feature extractor 206, label generator 208, and disorder classifiers 210a-210n (generally referred to as disorder classifiers 210 or a disorder classifier 210). The software routines of the pregnancy prediction engine 220 are executed by the analytics server 203 in the example system 200, though the software components of the pregnancy prediction engine 220 may be executed by any computing device comprising a processor capable of performing the operations of the pregnancy prediction engine 220 and by any number of such computing devices.

The data sources 201 may include any electronic computing devices or software components that contain, or provide, various types of data to the pregnancy prediction engine 220 of the analytics server 203. In some cases, a data source 201 includes a database (e.g., analytics databases 106, provider databases 118) containing EHRs or other types of data stored in non-transitory machine-readable storage memory. In some cases, a data source 201 includes a user device 216 that may store certain types of data in non-transitory storage, or may receive, via a user interface of the user device 216, user inputs indicating certain types of data. In some cases, a data source 201 is hosted at the analytics server 203 having the pregnancy prediction engine 220. Additionally or alternatively, in some cases, a data source 201 is hosted at a computing device (not shown) distinct from the analytics server 203 having the pregnancy prediction engine 220. In such cases, the data source 201 transmits the various types of data, via one or more networks, to the analytics server 203.

The user devices 216 (e.g., admin devices 103, patient devices 114, and provider devices 116) may include any type of computing device operated by users to interact with, or configure the functions of, the pregnancy prediction engine 220. The users operating the user devices 216 may include, for example, administrators of the system 200, clinicians (e.g., physicians, care workers, researchers), and patients, among others. The user may operate the user device 216 to enter various configuration inputs containing instructions for configuring the operations of the pregnancy prediction engine 220. The user may operate the user device 216 to provide various types of data input parameters for training, tuning, or deploying the pregnancy prediction engine 220.

The analytics server analytics server 203 (e.g., analytics server 102) may include any computing device(s) comprising hardware and software components capable of performing the features and functions described herein. The analytics server 203 may include, for example, non-transitory machine-readable storage memory and one or more processors for executing the software components of the pregnancy prediction engine 220. The analytics server 203 may receive the various data inputs and configuration inputs from the data sources 201 and user devices 216. The analytics server 203 may execute the operations of the pregnancy prediction engine 220 using the data input parameters and in accordance with the configurations.

The data ingestion engine 202 of the pregnancy prediction engine 220 includes software routines programmed to receive or retrieve the types of data from the data sources 201. The data ingestion engine 202 may be preprogrammed to obtain data from the data sources 201 at a preconfigured interval or in response to user instructions received as a configuration input or other user input from a user operating the user device 216. The data ingestion engine 202 may perform various functions for ingesting and preparing the data for use by the pregnancy prediction engine 220, such as collating, normalizing, organizing, parsing, and/or validating the data inputs, among other possible functions.

Certain data inputs or system configurations may be received at the data ingestion engine 202 (or other component of the system 200) from the user devices 216. For example, clinicians (e.g., physicians, healthcare workers, researchers) may indicate and configure the data sources 201, the types of data input parameters, or which disorder classifier 210 is relevant to which types of data parameters. In some cases, the users may conduct a review of, or configure threshold values that require assessments of, the types or amounts of data inputs obtained from the data sources 201. Non-limiting examples may include a review of, or threshold requirement for: data acquisition feasibility (e.g., volume of data, data transfer timeframes or throughput), a source population data, and containment of relevant data types, among others. In some implementations, the data ingestion engine 202 receives certain corrective inputs from the user devices 216. The data ingestion engine 202 may identify missing or inaccurate data values, and correct or update the data values according to the corrective inputs received from the user device 216.

For instance, although a data input may be available in the data sources 201, the users or the data ingestion engine 202 may determine whether the data input parameter will be readily available for a majority of patients (e.g., 80% complete or greater) for a given data source 201 (e.g., target databases for databases of target health data systems where disorder classifiers 210 will be applied; prior health data system databases where the disorder classifiers 210 were previously applied). Additionally or alternatively, the quality of a data input parameter in standard EHR database records may also be considered, where “data quality” may be an automatic or manual assessment of whether the particular type of data input parameter is fit to inform clinical decisions and involves evaluating accuracy and standardization of a given parameter, where “accuracy” means that the value of the parameter reflects the truth; and “standardization” means the parameter values will be standardized across clinical practices.

The data ingestion engine 202 (or other software component of the analytics server 203) may perform data augmentation operations on the data inputs obtained from the data sources 201. The data augmentation operations may generate additional types of data inputs or data points and/or generate synthetic training data for training machine-learning models of a machine-learning architecture of the pregnancy prediction engine 220.

In some cases, the data ingestion engine 202 may transform certain types of data health parameters. For instance, the data ingestion engine 202 may combine or parse certain data input parameters into a combined parameter. As an example, the data ingestion engine 202 may algorithmically combine height and weight measurements to compute or output a body mass index. In some cases, the data ingestion engine 202 may categorize continuous health parameters based on clinically relevant cutoffs or thresholds. For example, a continuous measure of body mass index may be categorized as “underweight,” “healthy weight,” “overweight,” or “obese” according to corresponding threshold values of the health parameters. As mentioned, in some implementations, the data ingestion engine 202 performs operations for dealing with missing values. In such cases, if the user device 216 inputs or the data ingestion engine 202 indicate there is an informative missing value, then the missing value will be encoded into a “missing” category, allowing the machine-learning models of the machine-learning architecture may learn from the missing values. If, however, the user device 216 or data ingestion engine 202 determines there is a non-informative missing value, then the data ingestion engine 202 may execute a function whereby the missing value is imputed using an imputation approach, such as K-nearest neighbors.

The pregnancy detector 204 of the pregnancy prediction engine 220 includes software routines for detecting, predicting, or otherwise identifying instances when a patient was pregnant based upon an analysis of the patient's care data, such as patient EHR data obtained from EHR databases (e.g., provider databases 118), among other potential data sources 201.

In some embodiments, the pregnancy detector 204 includes preconfigured functions and algorithms for analyzing the various types of data in the patient care data of the particular patient to identify pregnancy markers. The pregnancy markers include certain patient care data, or patterns of patient care data, indicative of a pregnancy within a given time interval. The functions of the pregnancy detector 204 may include applying various heuristics and comparative weights to the patient data to identify the pregnancy markers. The pregnancy detector 204 identifies or detects the instances of the pregnancy when the data of the pregnancy markers satisfy one or more threshold values. For example, the pregnancy detector 204 may query (e.g., execute a regular expression search) the patient care data to identify matching pregnancy indicators, such as certain metrics (e.g., blood work metrics), diagnostic or treatment codes, and data types (e.g., instances of sonogram images), among other types of data. In some configurations, the pregnancy detector 204 may detect an instance of pregnancy in response to determining that an amount of identified pregnancy indicators satisfy a threshold amount of matches. Additionally or alternatively, in some configurations, the pregnancy detector 204 may detect an instance of pregnancy in response to determining that the identified pregnancy indicators are associated with timestamps in the care data that satisfy one or more timing thresholds.

As mentioned, the administrator or clinician may operate the user device 216 to enter the configuration inputs to the analytics server 203 to configure operations of the pregnancy prediction engine 220. The configuration inputs may indicate, for example, the pregnancy indicators, the pregnancy markers, the pregnancy detection thresholds, the query parameters for identifying the pregnancy indicators or markers in the care data of the patients.

Optionally, in some embodiments, the pregnancy detector 204 may include machine-learning models and techniques, of a machine-learning architecture, trained for detecting an instance of pregnancy using the patient's care data. For example, the pregnancy detector 204 may extract a feature vector containing pregnancy indicator data and apply a pregnancy detector classifier (not shown) that computes a pregnancy likelihood score. The pregnancy detector 204 detects an instance of pregnancy when the pregnancy detector classifier determines that the pregnancy likelihood score satisfies a pregnancy detection threshold score.

The feature extractor 206 of the pregnancy prediction engine 220 includes software routines for identifying and extracting pregnancy care features or feature vectors. When the pregnancy detector 204 detects the pregnancy instance, the pregnancy detector 204 applies the feature extractor 206 on the care data and extracts the features from the patient's care records associated with the patient's pregnancy instance. For example, the pregnancy detector 204 may select the collection of one or more care data records having timestamps within a time interval threshold of the detected pregnancy instance. The pregnancy detector 204 may then extract the features and feature vector for the given pregnancy instance. Optionally, the feature extractor 206 may store the features into storage memory of the analytics server 203 or database (e.g., analytics database 106) of the system 200.

The feature extractor 206 may perform featurization operation of transforming varied forms of data inputs (from the data sources 201) to numerical data for use in the machine learning models of the machine-learning architecture of the pregnancy prediction engine 220. For example, for continuous time series data of fetal heart rate, the featurization operation involves extracting a finite set of measurements related to time and frequency.

In some embodiments, to improve model interpretability (i.e., for ease of explanation to clinicians), the user devices 216 provide user interface options for selecting and configuring the data input parameters for the data ingestion engine 202 and the features to be extracted by the feature extractor 206. In some cases, the variable selection techniques of the user device 216 or the pregnancy prediction engine 220 may create a “reduced model” with a subset of feature variables. In some implementations, the disorder classifiers 210 implement logistic regression for training on the extracted features. In such implementations, the disorder classifiers 210 may implement a least absolute shrinkage and selection (LASSO) approach, which shrinks the relative contributions of each feature variable towards zero depending on a preconfigured importance or weighted value. In some implementations, the disorder classifiers 210 may implement other types of machine-learning models, such as recursive feature elimination (RFE). In such embodiments, the RFE training operations may iteratively remove feature variables by, for example, fitting the given machine learning algorithm of the disorder classifier 210 on the training features of the training data, ranking features by weight importance, discarding one or more least important features (having comparatively lowest ranking weights), and re-fitting the model of the disorder classifier 210. The pregnancy prediction engine 220 may repeat the RFE training process until the amount of features remaining balance machine-learning model interpretability (e.g., fewer variables) against machine-learning model performance.

In some embodiments, the pregnancy prediction engine includes a label generator 208 that includes software routines for generating or otherwise associating extracted screening features and feature vectors with training labels, which indicate expected outcomes corresponding to the screening features. The expected outcomes indicate a “ground truth” for a given feature vector extracted from certain data values associated with a patient's pregnancy instance. In some circumstances, for example, the pregnancy detector 204 may misclassify an outcome label in a portion of the patient's data records (e.g., false positive, false negative). The label generator 208 generates the training labels for the feature vectors, indicating the ground truth of whether the feature vector extracted from the portion of patient data records is actually an instance of pregnancy and/or a particular disorder.

The label generator 208 may manually or automatically generate the training labels indicating the ground truth for screening feature vectors, extracted from the patient data inputs. In some cases, the user (e.g., administrator, clinician) may operate the user device 216 to enter configuration inputs that indicate the expected outcome of the training labels, which the pregnancy prediction engine 220 may feed into the label generator 208 to manually associate with the feature vectors. In some cases, the label generator 208 may be preconfigured to algorithmically determine whether the ground truth for the screening feature vector according to preconfigured heuristics and weights assigned to the types of data input parameters, where the label generator 208 may execute a distinct algorithm or thresholds from the pregnancy detector 204 for determining the ground truth of whether the patient's records indicate the pregnancy instance and/or the pregnancy disorders.

The training labels may be used to adjust the algorithms of the pregnancy detector 204 or feature extractor 206. As an example, the user may compare a generated training output of the pregnancy detector 204 against the training label indicating the ground truth to determine whether the pregnancy detector 204 properly detected a pregnancy instance in the patient's care data. The user may adjust the algorithm's functions, heuristics, types of data input parameters, weights, or thresholds to improve the accuracy of the pregnancy detector 204. As another example, the user may compare generated training outputs of the feature extractor 206 or disorder classifiers 210 against the training label indicating the ground truth to determine whether the feature extractor 206 extracted optimal screening features or the disorder classifiers 210 properly detected a given pregnancy disorder. The user may adjust the algorithm's functions, heuristics, types of data input parameters, weights, or thresholds of the feature extractor 206 to improve the accuracy of the screening features extracted from the patient's data records. The user device 216 or pregnancy prediction engine 220 may compute or determine which features provide the largest impact, reduce the features having a comparatively lower impact, and adjust the functions of the pregnancy detector 204, feature extractor 206, or disorder classifier 210. In some cases, for example, the pregnancy prediction engine 220 may implement a feature reduction approach (e.g. LASSO) to adjusting the pregnancy detector 204, feature extractor 206, or disorder classifiers 210.

As shown in FIG. 2, the label generator 208 is coupled to the feature extractor 206 in order to generate or otherwise associated the features or feature vectors with the training labels. Embodiments need not be so limited. Additionally or alternatively, in some embodiments, the label generator 208 is coupled to one or more data sources 201 or the data ingestion engine 202, such that the label generator 208 generates or otherwise associates the labels with the care data as obtained from the data sources 201 when the data ingestion engine 202 receives the care data from the data sources 201.

In some implementations, the disorder classifiers 210 may execute machine-learning models or techniques for training or tuning the hyperparameters or weights of the disorder classifiers 210 using a difference or loss function. The pregnancy prediction engine 220 may determine a difference or loss (e.g., level of error) based upon comparing a predicted outcome of the disorder classifier 210 compared against the training label indicating the expected outcome.

The disorder classifiers 210 of the pregnancy prediction engine 220 for detecting a pregnancy disorder in the patient's care data. The machine-learning architecture of the pregnancy prediction engine 220 comprises any number of disorder classifiers 210 to classify particular pregnancy disorders in the patient care data, where each disorder classifier 210 is programmed and trained to detect whether the patient care data indicates a particular type of pregnancy disorder. In some implementations, the disorder classifiers 210 include functions of a binary classifier. Additionally or alternatively, in some implementations, one or more of the disorder classifiers 210 include functions of a multi-class classifier. For example, a first disorder classifier 210a includes a hypertensive disorder detector trained to detect and classify whether the patient care data indicates a hypertensive disorder. As another example, a second disorder classifier 210b includes a GDM detector trained to detect and classify whether the patient care data indicates an instance of GDM. Embodiments may include additional or alternative examples of disorder classifiers 210, as described herein, implemented in the pregnancy prediction engine 220. In operation, the pregnancy prediction engine 220 applies the disorder classifiers 210 on the screening feature vectors to output a detection outcome. The disorder classifier 210 generates a disorder probability score, indicating the likelihood or probability that the disorder is indicated in the patient care data. The disorder classifier 210 detects the disorder, in response to determining that the disorder probability score satisfies a detection threshold score.

As mentioned, the disorder classifiers 210 may execute machine-learning models or techniques for training or tuning the hyperparameters or weights of the disorder classifiers 210 using the difference or loss function. The pregnancy prediction engine 220 may determine a difference or loss (e.g., level of error) based upon comparing a predicted outcome of the disorder classifier 210 compared against the training label indicating the expected outcome.

In some embodiments, the pregnancy prediction engine 220 may employ data augmentation operations for training the disorder classifiers 210 based upon one or more temporal features included in, or otherwise associated with, the feature vectors used for training the disorder classifiers 210. In many circumstances, certain types of data may not always be available to the pregnancy prediction engine 220 at deployment inference time. Due to real-world delays, there may be lag time between when a test is performed or sample is taken and when the measurement or metrics are included into the patient care data. The disorder classifiers 210 are trained according to distribution curves plotting the generated outputs for each of the training input vectors. The real-world delays in the data may distort the results of the disorder classifiers 210. At interference time, the presence or lack of certain types of data inputs used for extracting the patient's feature vector at a given point in the patient's pregnancy could cause the disorder classifier 210 to predict a skewed outcome relative to the disorder classifier's 210 distribution and potentially an inaccurate outcome relative to the patient's real-world condition. To address these issues, a data augmentation process associates timestamps with the training data or feature vectors at training time for robustness. The pregnancy prediction engine 220 may include or associate one or more timestamps (e.g., when a data input value was generated as a measurement; when the data input value was available to the pregnancy prediction engine 220 in the patient care data). These timestamps may be associated with certain data input values of the training data and/or associated with the training feature vectors as a whole before feeding such feature vectors to the disorder classifiers 210 at training time.

### EXAMPLE EMBODIMENTS

### Example 1: Prediction of Hypertensive Disorders of Pregnancy (HDPs)

The incidence of HDPs has doubled in the United States from 2008 to 2019. Strikingly, 24% of maternal deaths that occurred during delivery hospitalization had a diagnosis code for HDP documented. Early identification of patients who would benefit from HDP interventions may reduce the incidence of HDP by allowing physicians initiate lifestyle-based interventions or to prescribe prophylactic or intervention treatments. Additionally, current ACOG risk criteria can be improved upon by using machine learning.

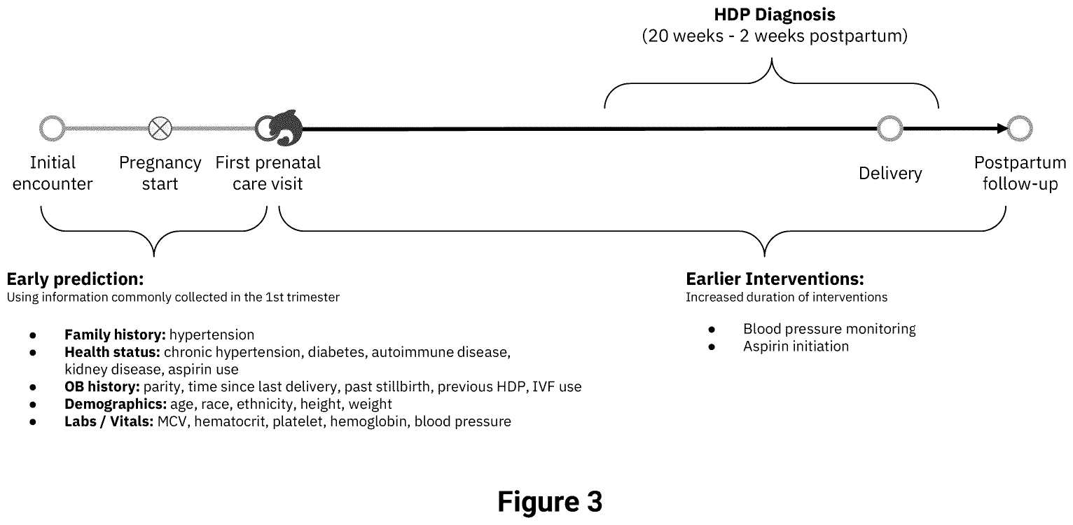

FIG. 3 depicts an exemplary timeline for early prediction and intervention for HDP. As shown on the timeline, early prediction may involve information collected during the first trimester of pregnancy. As non-limiting examples, family history of hypertension, chronic hypertension, diabetes, autoimmune disease, kidney disease, aspirin use, parity, last time since delivery, past stillbirth, previous HDP, IVF use, age, race, ethnicity, height, weight, MCV, hematocrit, platelet, hemoglobin, blood pressure, etc. may be included as health parameters which are collected and which may be useful for predictive methods. Also, as shown on the timeline of FIG. 3, HDP diagnosis may not occur under current guidelines until 20 weeks into pregnancy, up until 2 weeks postpartum. This leaves a significant gap in time where the patient may have detrimental HDP development. With early detection models, earlier intervention may be prescribed such as blood pressure monitoring or aspirin (or other drug) intervention. By this manner, harmful effects of HDP on the patient and child may be reduced or mitigated.

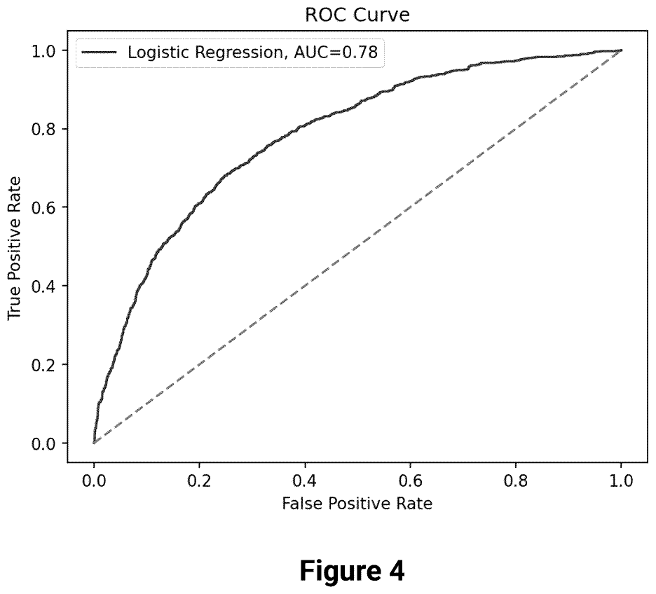

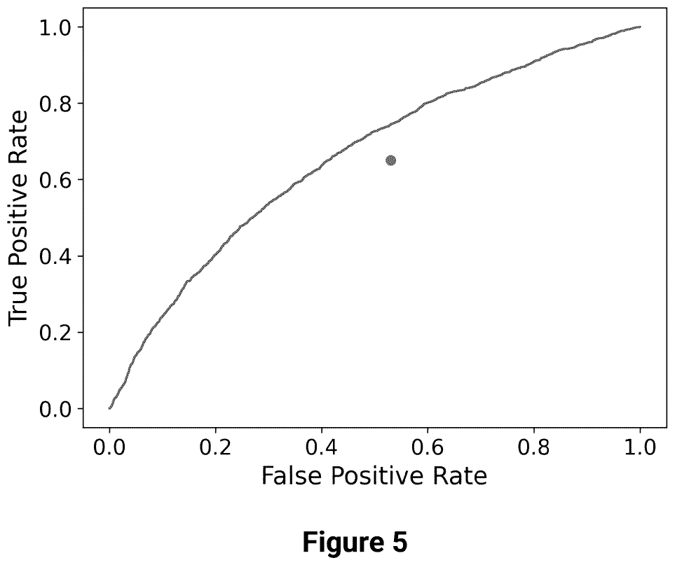

As depicted in FIG. 4, a machine learning model was trained and validated on internal data. The ROC curve of FIG. 4 shows an AUC of 0.78, indicating that the model performs well in predicting HDP in the internal validation cohort. Moreover, as shown in FIG. 5 the model performs well in an external cohort of nulliparous pregnancies, and even outperforms the ACOG checklist (USPSTF aspirin initiation guidelines). Based upon this surprising result, it has been demonstrated that the systems and methods herein may provide for both earlier (i.e., first trimester) and more reliable disease prediction than existing methods. The study population included 18,456 pregnancies from a large academic medical center in the midwestern United States and the model type used in this example was a logistic regression model.

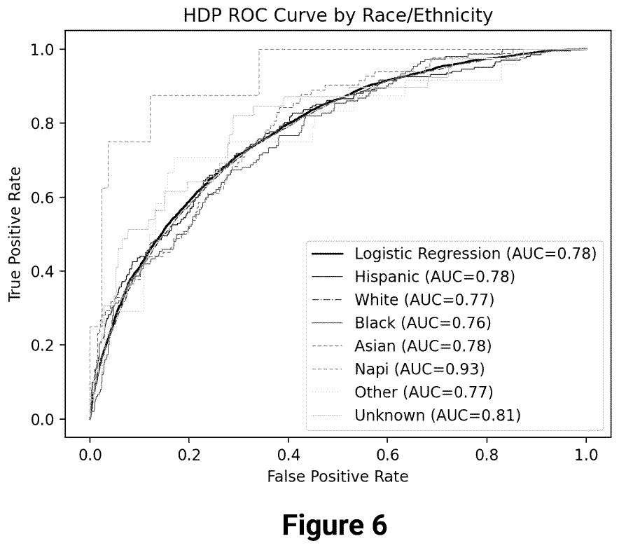

The HDP model was also tested for performance differences across different races/ethnicities, as depicted in FIG. 6. No differences in model performance by race/ethnicity were observed, and AUC values ranged from 0.77 to 0.93 indicating strong performance across the tested categories.

### Example 2: Prediction of Gestational Diabetes (GDM)

Between 2016 to 2020, the incidence of GDM increased by 30%. Pregnancies complicated by GDM have an increased risk of preterm birth, macrosomia, and large for gestational age infants. Further, 10% of women with GDM develop Type II diabetes soon after delivery.

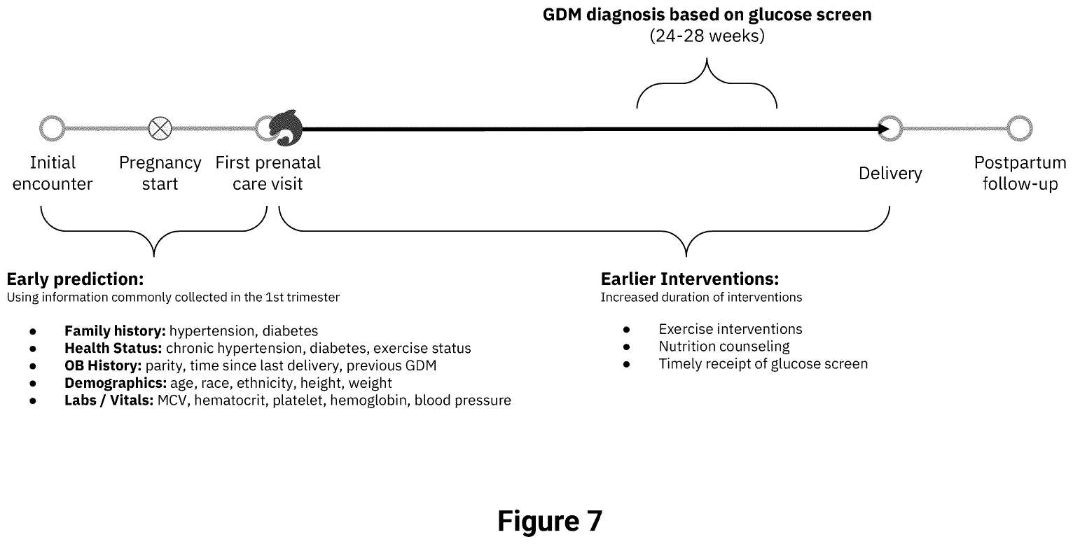

FIG. 7 depicts an exemplary timeline for early prediction and intervention for GDM. As shown on the timeline, early prediction may involve information collected during the first trimester of pregnancy. As non-limiting examples, family history of hypertension, chronic hypertension, diabetes, exercise status, parity, last time since delivery, previous GDM, age, race, ethnicity, height, weight, MCV, hematocrit, platelet, hemoglobin, blood pressure, etc. may be included as health parameters which are collected and which may be useful for predictive methods. Also as shown on the timeline of FIG. 7, GDM diagnosis may not occur under current guidelines until 24 weeks into pregnancy, up until 28 weeks into pregnancy. This leaves a significant gap in time where the patient may have detrimental GDM development. With early detection models, earlier intervention may be prescribed such as exercise interventions, nutrition counseling, and timely receipt of glucose screens. By this manner, harmful effects of GDM on the patient and child may be reduced or mitigated.

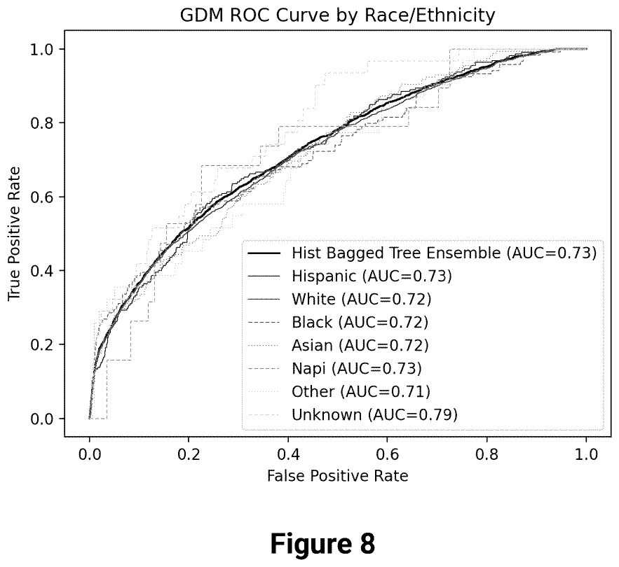

As depicted in FIG. 8, a machine learning model was trained and validated upon internal data. The ROC curve of FIG. 8 shows an AUC of 0.73, indicating that the model performs well in predicting GDM in the internal validation cohort. The study population included 23,202 pregnancies from a large academic medical center in the midwestern United States. Patients having pregestational diabetes were excluded from the model. The model type utilized in this example was a histogram-based bagged tree ensemble.

### Example 3: Prediction of Excessive Gestational Weight Gain (eGWG)

Nearly 50% of pregnancies in the United States exceed gestational weight gain (GWG) targets, which may lead to significantly higher risk of HDP, GDM, and long-term adverse metabolic outcomes. Identifying individuals at highest risk for eGWG early in pregnancy offers opportunities for counseling and behavioral interventions to reduce weight gain.

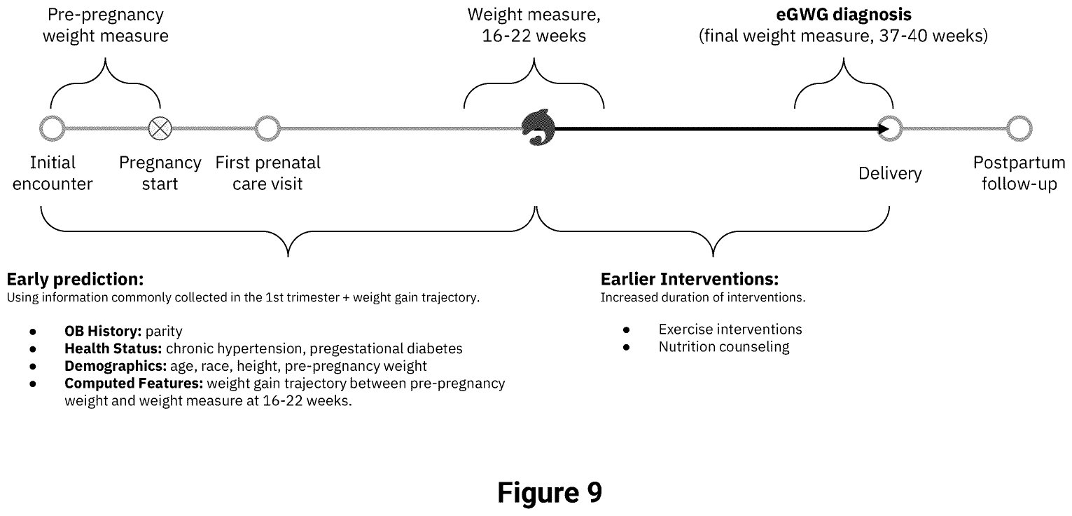

FIG. 9 depicts an exemplary timeline for early prediction and intervention for eGWG. As shown on the timeline, early prediction may involve information collected during the first trimester of pregnancy. As non-limiting examples, OB history of parity, chronic hypertension, pregestational diabetes, age, race, height, weight, pre-pregnancy weight, etc. may be included as health parameters which are collected and which may be useful for predictive methods. Also as shown on the timeline of FIG. 9, eGWG diagnosis may not occur under current guidelines until 37 weeks into pregnancy, up until 40 weeks into pregnancy. This leaves a significant gap in time where the patient may have detrimental eGWG development. With early detection models, earlier intervention may be prescribed such as exercise interventions and nutrition counseling. By this manner, harmful effects of eGWG on the patient and child may be reduced or mitigated.

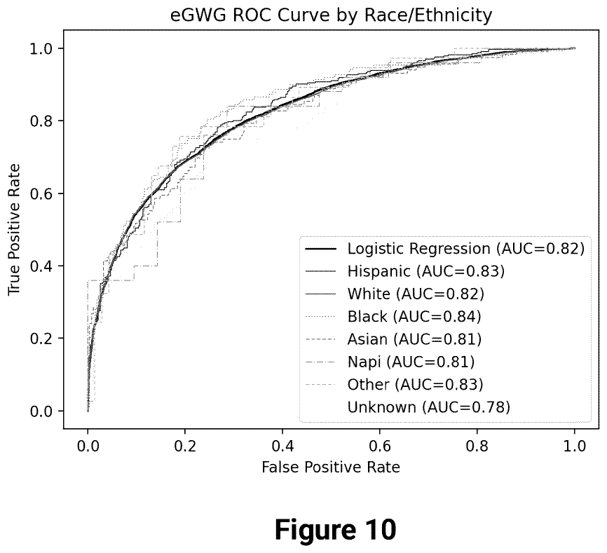

As depicted in FIG. 10, a machine learning model was trained and validated on internal data. The ROC curve of FIG. 10 shows an AUC of 0.82, indicating that the model performs well in predicting eGWG in the internal validation cohort. The study population included 10,867 pregnancies from a large academic medical center in the midwestern United States. Patients having preterm delivery or missing weight measurements were excluded from the model. The model type utilized in this example was a logistic regression.

### Example 4: Comparison of Model Performances

As described herein models for hypertensive disorders of pregnancy, gestational diabetes, and excessive gestational weight gain were developed and shown to effectively predict these pregnancy disorders. A comparison of these developed models is shown below in Table 2:

| | Training | Preva- | | Sensitivity* | Specificity* |
|---|---|---|---|---|---|
| | Sample | lence | | true | true |
| Outcome | Size | (%) | AUC | positives | negatives |
| Hypertensive | 18,456 | 18.5% | AUC = | 0.70 | 0.71 |
| disorders of | | | 0.78 | | |
| pregnancy | | | | | |
| Gestational | 23,202 | 10.0% | AUC = | 0.65 | 0.65 |
| diabetes | | | 0.73 | | |
| Excessive | 10,867 | 55.4% | AUC = | 0.75 | 0.74 |
| gestational | | | 0.82 | | |
| weight gain | | | | | |

*The given values of TABLE 2 for sensitivity and specificity correspond to the maximum value of sensitivity + specificity across all probability cutoffs.

### Example 5: Optimized Health Parameter Sets for Outcome Prediction

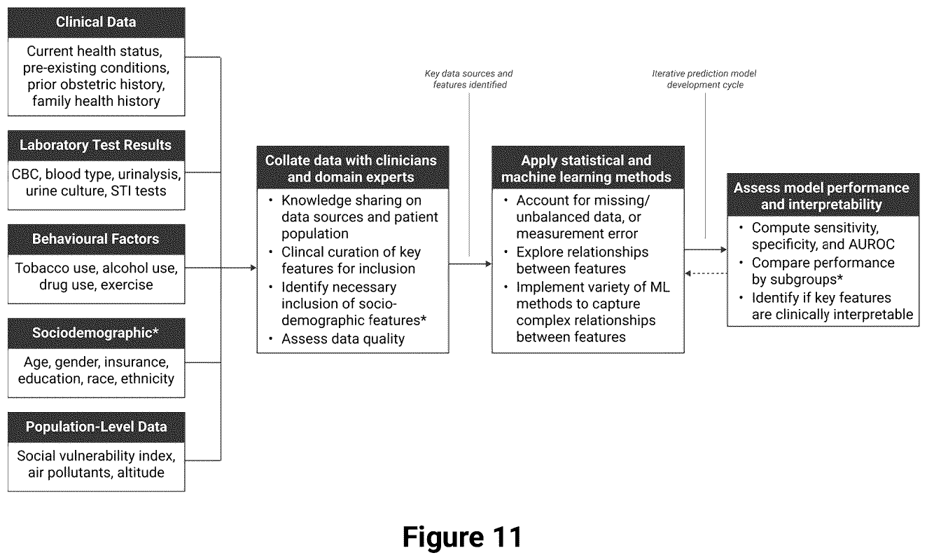

As described herein, robust predictive model development includes data selection and training. The present invention includes the discovery of groupings of input health parameters which provide high-performing predictive models for prediction of pregnancy disorders. FIG. 11 depicts operational steps of predictive machine learning method, generally. For example, these groupings of health parameters may advantageously provide for models having an AUC of at least about 0.7, and in many cases higher. In embodiments herein, the models may be limited to one of the groupings of input health parameters (that is, the input health parameters could consist of a grouping). In embodiments herein, the models may include, or alternatively may consist of, about 80% of the parameters in a particular grouping of input health parameters. Exemplary advantageous embodiments including input health parameter grouping sets are shown in Table 3 below:

TABLE 3
Exemplary machine learning models for early prediction of pregnancy disorders.

| | | | | Internal |
|---|---|---|---|---|
| | Outcome in | | | performance |
| | Training | | | metrics |
| # | Dataset | Input Health Parameters | Model Architecture | AUC (C.I.) |
| 1 | Hypertensive | age, BMI, time since last | multi-layer perceptron | 0.733456 |
| | disorders of | delivery, autoimmune | (MLP) neural network | (0.724655, |
| | pregnancy | condition, kidney disease, | | 0.742220) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy | | |
| 2 | Hypertensive | age, BMI, time since last | logistic regression | 0.736911 |
| | disorders of | delivery, autoimmune | (LR) | (0.728964, |
| | pregnancy | condition, kidney disease, | | 0.745143) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy | | |
| 3 | Hypertensive | age, BMI, time since last | ensemble (histogram | 0.735155 |
| | disorders of | delivery, autoimmune | gradient boosting, | (0.726578, |
| | pregnancy | condition, kidney disease, | Adaboost, LR, MLP) | 0.744126) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy | | |
| 4 | Hypertensive | age, BMI, time since last | histogram-based | 0.737919 |
| | disorders of | delivery, autoimmune | gradient boosting | (0.729360, |
| | pregnancy | condition, kidney disease, | | 0.746728) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy | | |
| 5 | Hypertensive | age, BMI, time since last | adaptive boosting | 0.732983 |
| | disorders of | delivery, autoimmune | (Adaboost) | (0.723955, |
| | pregnancy | condition, kidney disease, | | 0.741662) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy | | |
| 6 | Hypertensive | age, BMI, time since last | multi-layer perceptron | 0.775971 |
| | disorders of | delivery, autoimmune | (MLP) neural network | (0.767592, |
| | pregnancy | condition, kidney disease, | | 0.783993) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy, systolic BP, | | |
| | | diastolic BP, hemoglobin, | | |
| | | hematocrit, platelet, MCV | | |
| 7 | Hypertensive | age, BMI, time since last | logistic regression | 0.777526 |
| | disorders of | delivery, autoimmune | (LR) | (0.769511, |
| | pregnancy | condition, kidney disease, | | 0.785500) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy, systolic BP, | | |
| | | diastolic BP, hemoglobin, | | |
| | | hematocrit, platelet, MCV | | |
| 8 | Hypertensive | age, BMI, time since last | ensemble (histogram | 0.772440 |
| | disorders of | delivery, autoimmune | gradient boosting, | (0.764205, |
| | pregnancy | condition, kidney disease, | Adaboost, LR, MLP) | 0.780615) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy, systolic BP, | | |
| | | diastolic BP, hemoglobin, | | |
| | | hematocrit, platelet, MCV | | |
| 9 | Hypertensive | age, BMI, time since last | histogram-based | 0.776895 |
| | disorders of | delivery, autoimmune | gradient boosting | (0.768957, |
| | pregnancy | condition, kidney disease, | | 0.785179) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy, systolic BP, | | |
| | | diastolic BP, hemoglobin, | | |
| | | hematocrit, platelet, MCV | | |
| 10 | Hypertensive | age, BMI, time since last | adaptive boosting | 0.771841 |
| | disorders of | delivery, autoimmune | (Adaboost) | (0.763225, |
| | pregnancy | condition, kidney disease, | | 0.779710) |
| | | prior stillbirth, | | |
| | | pregestational diabetes, | | |
| | | prior HDP, chronic | | |
| | | hypertension, race, | | |
| | | Hispanic ethnicity, | | |
| | | multigestation, nulliparity, | | |
| | | family history of | | |
| | | hypertension, IVF for this | | |
| | | pregnancy, systolic BP, | | |
| | | diastolic BP, hemoglobin, | | |
| | | hematocrit, platelet, MCV | | |
| 11 | Excessive | weight gain slope, age, | logistic regression | 0.823087 |
| | gestational | BMI, nulliparity | (LR) | (0.815038, |
| | weight gain | | | 0.830671) |
| 12 | Excessive | weight gain slope, age, | logistic regression | 0.823087 |
| | gestational | BMI, nulliparity, chronic | (LR) | (0.815552, |
| | weight gain | hypertension, | | 0.830741) |
| | | pregestational diabetes | | |
| 13 | Gestational | age, BMI, race, Hispanic | logistic regression | 0.702259 |
| | diabetes | ethnicity, cardiovascular | (LR) | (0.689333, |
| | (based on | disease, previous | | 0.716446) |
| | ICD-10 | gestational diabetes, | | |
| | diagnostic | insulin resistance, | | |
| | code and | polycystic ovarian | | |
| | glucose | syndrome, chronic | | |
| | tolerance test | hypertension, family | | |
| | results) | history of diabetes | | |
| 14 | Gestational | age, BMI, race, Hispanic | histogram-based | 0.699936 |
| | diabetes | ethnicity, cardiovascular | gradient boosting | (0.687170, |
| | (based on | disease, previous | | 0.713544) |
| | ICD-10 | gestational diabetes, | | |
| | diagnostic | insulin resistance, | | |
| | code and | polycystic ovarian | | |
| | glucose | syndrome, chronic | | |
| | tolerance test | hypertension, family | | |
| | results) | history of diabetes | | |
| 15 | Gestational | age, BMI, race, Hispanic | histogram-based | 0.699333 |
| | diabetes | ethnicity, cardiovascular | gradient boosting | (0.687081, |
| | (based on | disease, previous | (bagged) | 0.712353) |
| | ICD-10 | gestational diabetes, | | |
| | diagnostic | insulin resistance, | | |
| | code and | polycystic ovarian | | |
| | glucose | syndrome, chronic | | |
| | tolerance test | hypertension, family | | |
| | results) | history of diabetes | | |
| 16 | Gestational | age, BMI, race, Hispanic | logistic regression | 0.721883 |
| | diabetes | ethnicity, cardiovascular | (LR) | (0.710420, |
| | (based on | disease, previous | | 0.733458) |
| | ICD-10 | gestational diabetes, | | |
| | diagnostic | insulin resistance, | | |
| | code only) | polycystic ovarian | | |
| | | syndrome, chronic | | |
| | | hypertension, family | | |
| | | history of diabetes | | |
| 17 | Gestational | age, BMI, race, Hispanic | histogram-based | 0.718770 |
| | diabetes | ethnicity, cardiovascular | gradient boosting | (0.706615, |
| | (based on | disease, previous | | 0.730771) |
| | ICD-10 | gestational diabetes, | | |
| | diagnostic | insulin resistance, | | |
| | code only) | polycystic ovarian | | |
| | | syndrome, chronic | | |
| | | hypertension, family | | |
| | | history of diabetes | | |
| 18 | Gestational | age, BMI, race, Hispanic | histogram-based | 0.721464 |
| | diabetes | ethnicity, cardiovascular | gradient boosting | (0.710604, |
| | (based on | disease, previous | (bagged) | 0.732802) |
| | ICD-10 | gestational diabetes, | | |
| | diagnostic | insulin resistance, | | |
| | code only) | polycystic ovarian | | |
| | | syndrome, chronic | | |
| | | hypertension, family | | |
| | | history of diabetes | | |
| 19 | Gestational | age, BMI, race, Hispanic | logistic regression | 0.674198 |
| | diabetes | ethnicity, cardiovascular | (LR) | (0.659128, |
| | (based on | disease, previous | | 0.689273) |
| | glucose | gestational diabetes, | | |
| | tolerance test | insulin resistance, | | |
| | values only) | polycystic ovarian | | |
| | | syndrome, chronic | | |
| | | hypertension, family | | |
| | | history of diabetes | | |
| 20 | Gestational | age, BMI, race, Hispanic | histogram-based | 0.673685 |
| | diabetes | ethnicity, cardiovascular | gradient boosting | (0.658367, |
| | (based on | disease, previous | (bagged) | 0.688217) |
| | glucose | gestational diabetes, | | |
| | tolerance test | insulin resistance, | | |
| | values only) | polycystic ovarian | | |
| | | syndrome, chronic | | |
| | | hypertension, family | | |
| | | history of diabetes | | |
| 21 | Gestational | age, BMI, race, Hispanic | logistic regression | 0.710211 |
| | diabetes | ethnicity, cardiovascular | (LR) | (0.697472, |
| | (based on | disease, previous | | 0.722881) |
| | ICD-10 | gestational diabetes, | | |
| | diagnostic | insulin resistance, | | |
| | code and | polycystic ovarian | | |
| | glucose | syndrome, chronic | | |
| | tolerance test | hypertension, family | | |
| | results) | history of diabetes, | | |
| | | hemoglobin, hematocrit, | | |
| | | platelet, MCV | | |
| 22 | Gestational | age, BMI, race, Hispanic | histogram-based | 0.701355 |
| | diabetes | ethnicity, cardiovascular | gradient boosting | (0.688002, |
| | (based on | disease, previous | | 0.714084) |
| | ICD-10 | gestational diabetes, | | |
| | diagnostic | insulin resistance, | | |
| | code and | polycystic ovarian | | |
| | glucose | syndrome, chronic | | |
| | tolerance test | hypertension, family | | |
| | results) | history of diabetes, | | |
| | | hemoglobin, hematocrit, | | |
| | | platelet, MCV | | |
| 23 | Gestational | age, BMI, race, Hispanic | histogram-based | 0.704528 |
| | diabetes | ethnicity, cardiovascular | gradient boosting | (0.690935, |
| | (based on | disease, previous | (bagged) | 0.717398) |
| | ICD-10 | gestational diabetes, | | |
| | diagnostic | insulin resistance, | | |
| | code and | polycystic ovarian | | |
| | glucose | syndrome, chronic | | |
| | tolerance test | hypertension, family | | |
| | results) | history of diabetes, | | |
| | | hemoglobin, hematocrit, | | |
| | | platelet, MCV | | |
| 24 | Gestational | age, BMI, race, Hispanic | logistic regression | 0.731365 |
| | diabetes | ethnicity, cardiovascular | (LR) | (0.718790, |
| | (based on | disease, previous | | 0.742401) |
| | ICD-10 | gestational diabetes, | | |
| | diagnostic | insulin resistance, | | |
| | code only) | polycystic ovarian | | |
| | | syndrome, chronic | | |
| | | hypertension, family | | |
| | | history of diabetes, | | |
| | | hemoglobin, hematocrit, | | |
| | | platelet, MCV | | |
| 25 | Gestational | age, BMI, race, Hispanic | histogram-based | 0.726931 |
| | diabetes | ethnicity, cardiovascular | gradient boosting | (0.716079, |
| | (based on | disease, previous | | 0.737492) |
| | ICD-10 | gestational diabetes, | | |
| | diagnostic | insulin resistance, | | |
| | code only) | polycystic ovarian | | |
| | | syndrome, chronic | | |
| | | hypertension, family | | |
| | | history of diabetes, | | |
| | | hemoglobin, hematocrit, | | |
| | | platelet, MCV | | |
| 26 | Gestational | age, BMI, race, Hispanic | histogram-based | 0.728229 |
| | diabetes | ethnicity, cardiovascular | gradient boosting | (0.716070, |
| | (based on | disease, previous | (bagged) | 0.739828) |
| | ICD-10 | gestational diabetes, | | |
| | diagnostic | insulin resistance, | | |
| | code only) | polycystic ovarian | | |
| | | syndrome, chronic | | |
| | | hypertension, family | | |
| | | history of diabetes, | | |
| | | hemoglobin, hematocrit, | | |
| | | platelet, MCV | | |
| 27 | Gestational | age, BMI, race, Hispanic | logistic regression | 0.682409 |
| | diabetes | ethnicity, cardiovascular | (LR) | (0.668731, |
| | (based on | disease, previous | | 0.696893) |
| | glucose | gestational diabetes, | | |
| | tolerance test | insulin resistance, | | |
| | values only) | polycystic ovarian | | |
| | | syndrome, chronic | | |
| | | hypertension, family | | |
| | | history of diabetes, | | |
| | | hemoglobin, hematocrit, | | |
| | | platelet, MCV | | |
| 28 | Gestational | age, BMI, race, Hispanic | histogram-based | 0.679084 |
| | diabetes | ethnicity, cardiovascular | gradient boosting | (0.663777, |
| | (based on | disease, previous | (bagged) | 0.693167) |
| | glucose | gestational diabetes, | | |
| | tolerance test | insulin resistance, | | |
| | values only) | polycystic ovarian | | |
| | | syndrome, chronic | | |
| | | hypertension, family | | |
| | | history of diabetes, | | |
| | | hemoglobin, hematocrit, | | |
| | | platelet, MCV | | |
| 29 | Hypertensive | 143 predictors (not all | random forest | 0.73 |
| | disorders of | listed here but they span | | (0.70, 0.75) |
| | pregnancy | demographic and health | | |
| | | factors, | | |
| | | laboratory results, medical | | |
| | | conditions and diagnosis, | | |
| | | and family health history) | | |
| 30 | Hypertensive | hemoglobin, hematocrit, | random forest | 0.71 |
| | disorders of | MCV, blood type, | | (0.68, 0.74) |
| | pregnancy | chlamydial screen, nuchal | | |
| | | translucency, urine | | |
| | | culture, depression, | | |
| | | anxiety, age, BMI, tobacco | | |
| | | use, pre-pregnancy weight, | | |
| | | pregestational diabetes, | | |
| | | illicit drug use, chronic | | |
| | | hypertension, asthma, | | |
| | | migraines, urinary tract | | |
| | | infections, yeast | | |
| | | infections, family history | | |
| | | of preterm birth, family | | |
| | | history of low birth weight, | | |
| | | family history of | | |
| | | hypertensive disorders of | | |
| | | pregnancy, max BP first | | |
| | | visit | | |
| 31 | Excessive | rate of mid-pregnancy | logistic regression | 0.846 |
| | gestational | gestational weight gain, | (LR) | (0.825, 0.867) |
| | weight gain | age, BMI, chronic | | |
| | | hypertension, | | |
| | | pre-gestational diabetes | | |
| 32 | Excessive | rate of mid-pregnancy | generalized additive | 0.850 |
| | gestational | gestational weight gain, | model (GAM) | (0.830, 0.871) |
| | weight gain | age, BMI, chronic | | |
| | | hypertension, | | |
| | | pre-gestational diabetes | | |
| 33 | Excessive | rate of mid-pregnancy | random forest | 0.849 |
| | gestational | gestational weight gain, | | (0.828, 0.870) |
| | weight gain | age, BMI, chronic | | |
| | | hypertension, | | |
| | | pre-gestational diabetes | | |
| 34 | Gestational | age, BMI, high blood | logistic regression | 0.74 |
| | diabetes | pressure, family member | (LR) | (0.66, 0.82) |
| | (based on | with diabetes, valvular | | |
| | glucose | heart disease, coronary | | |
| | tolerance test | artery disease, PCOS | | |
| | values only) | | | |
| 35 | Gestational | age, BMI, height, pre- | LR + LASSO | 0.76 |
| | diabetes | pregnancy weight, | | (0.68, 0.84) |
| | (based on | gravidity, tobacco use, | | |
| | glucose | insurance type, mental | | |
| | tolerance test | health condition, | | |
| | values only) | hemoglobin, hematocrit, | | |
| | | MCV, platelet count, | | |
| | | blood type, Rh factor, | | |
| | | antibody screen, urine | | |
| | | culture, gonorrheal screen, | | |
| | | chlamydial screen, | | |
| | | syphilis, HIV, nuchal | | |
| | | translucency, PAPP-A, | | |
| | | family member with | | |
| | | diabetes, high blood | | |
| | | pressure, asthma, seizure, | | |
| | | migraines, | | |
| | | hyperthyroidism, | | |
| | | hypothyroidism, valvular | | |
| | | heart disease, coronary | | |
| | | artery disease, cardiac | | |
| | | arrhythmia, kidney | | |
| | | disease, history of blood | | |
| | | clots, PCOS, bacterial | | |
| | | vaginosis | | |
| 36 | Gestational | age, BMI, height, pre- | random forest | 0.77 |
| | diabetes | pregnancy weight, | | (0.69, 0.85) |
| | (based on | gravidity, tobacco use, | | |
| | glucose | insurance type, mental | | |
| | tolerance test | health condition, | | |
| | values only) | hemoglobin, hematocrit, | | |
| | | MCV, platelet count, | | |
| | | blood type, Rh factor, | | |
| | | antibody screen, urine | | |
| | | culture, gonorrheal screen, | | |
| | | chlamydial screen, | | |
| | | syphilis, HIV, nuchal | | |
| | | translucency, PAPP-A, | | |
| | | family member with | | |
| | | diabetes, high blood | | |
| | | pressure, asthma, seizure, | | |
| | | migraines, | | |
| | | hyperthyroidism, | | |
| | | hypothyroidism, valvular | | |
| | | heart disease, coronary | | |
| | | artery disease, cardiac | | |
| | | arrhythmia, kidney | | |
| | | disease, history of blood | | |
| | | clots, PCOS, bacterial | | |
| | | vaginosis | | |
| 37 | Small for | age, race, Hispanic | stochastic gradient | 0.80 |
| | gestational | ethnicity, insurance type, | boosting | (0.80, 0.81) |
| | age | delivery gestational age, | | |
| | | parity, body mass index, | | |
| | | gestational weight gain, | | |
| | | pregestational weight, | | |
| | | chronic hypertension, | | |
| | | pregestational/gestational | | |
| | | diabetes, hypertensive | | |
| | | disorders of pregnancy, | | |
| | | neonatal sex | | |

### Example 6: Prediction of HDP with High Confidence in the First Trimester

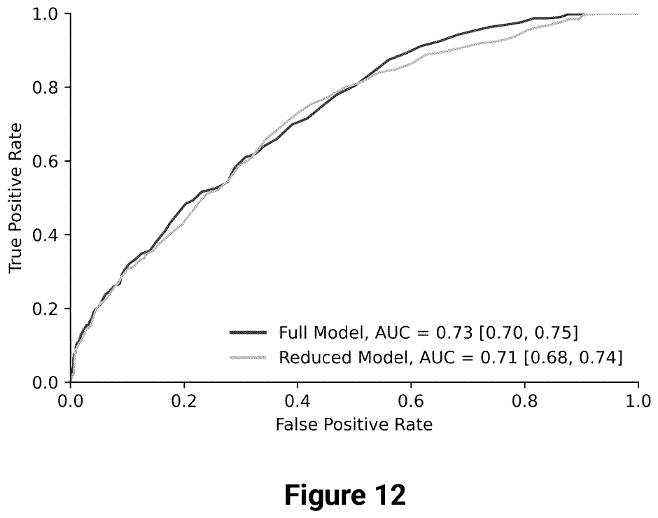

As shown in FIG. 12, a machine learning model was trained for effective prediction of HDP in the first trimester. The full model included the key health parameters BMI, pre-pregnancy weight, SBP, DBP, hypertension, asthma, migraines, UTI, anemia, mental health, gonorrhea, chlamydia, yeast infection, bacterial vaginosis, diabetes, hemoglobin, hematocrit, MCV, platelet, blood type, urine culture, nuchal translucency, tobacco use, age, and insurance.

In this study, the study data utilized was from a publicly available dataset of nulliparous pregnancies. The primary diagnosis was a diagnosis with any hypertensive disorder of pregnancy (HDP) between 20 weeks gestation and two weeks postpartum, an outcome readily available in the dataset. HDP included diagnosis of gestational hypertension, preeclampsia with/without severe features, superimposed preeclampsia with/without severe features, and eclampsia.

In this study, 207 predictor health parameters were included in building the full model, and the model did not include information on maternal race and ethnicity in the training of our prediction model. However, maternal race and ethnicity were used to evaluate model performance by race and ethnicity in stratified analyses. Additionally, missingness was determined to be informative for the outcome of HDP and was included in the analysis. The final sample size was 9,124. Any predictors in either set that had missing values were specifically encoded as “−10000” for individuals for which the value was unknown, allowing the subsequent models to learn even in the absence of a predictor.

To train the model, 80% of patients were randomly allocated to a training set and 20% of patients were randomly allocated to a holdout training set. Balanced class weights were utilized to account for imbalance in HDP diagnosis, which ensures that misclassification in the minority class (HDP diagnosis) will be penalized similarly to the majority class (no HDP diagnosis). For the outcome of HDP diagnosis, a random forest model was developed to differentiate between individuals with and without HDP with the full set of predictors/parameters. To find optimal hyperparameters for the model, such as the number of decision trees to include, a gridsearch with 3-fold cross validation was used with sensitivity as the scoring metric. This yielded a maximum tree depth of 50, a minimum number of samples per leaf node of 1, and a minimum number of samples required to split, and an internal node of 2. To improve model interpretability, a reduced model with 30 predictors was created using recursive feature elimination.

The full model performed well with an AUC of 0.73 (95% CI: 0.70, 0.75). However, as explained, full models produce output variables which may be difficult for healthcare workers to understand. In many cases, understanding of these variables may be improved by reducing the model to limit the number of output variables as described herein. As shown in FIG. 12, reducing the model had a limited effect on the performance as evaluated by the AUC value of 0.71 (95% CI: 0.68, 0.74), indicating that the models are robust enough to undergo reducing procedures while retaining acceptable performance.

### Example 7: HDP Model Outperforms USPSTF Guidelines

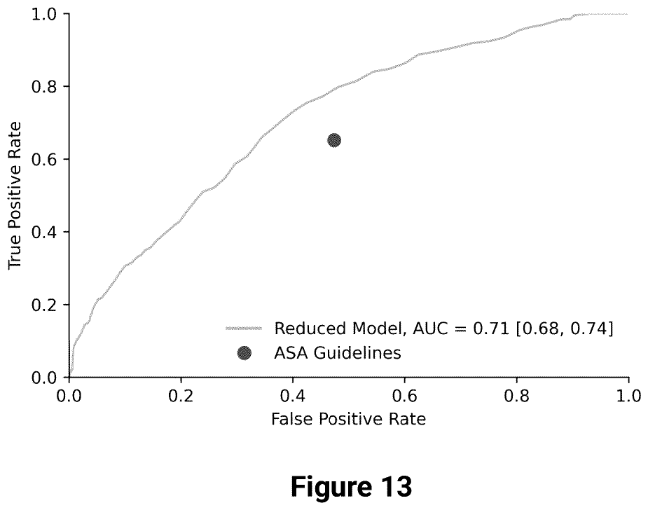

In pregnant patients at risk for cardiovascular issues, USPSTF guidelines provide for when physicians should initiate aspirin administration. FIG. 13 depicts the reduced HDP model of Example 6 compared to the aspirin administration guidelines. When set at the same specificity level as the USPSTF guidelines (53%), the reduced HDP model performs at a sensitivity of 80%. Compared to USPSTF guidelines, this translates to an additional 15 HDP patients for every 100 HDP patients correctly initiated on aspirin. This result indicates failures in the traditional guidelines-15% of patients who would have benefitted from aspirin administration would not be treated and may suffer preventable health consequences as a result. Additionally, when set at the same level of sensitivity, the reduced model had a specificity of 0.65. This translates to 12 additional non-HDP patients for every 100 non-HDP patients who would not be initiated on aspirin using the current model compared to traditional guidelines. Therefore, the machine learning models and related methods described herein provide for unprecedented prediction of pregnancy disorder outcomes to direct disease intervention and improve patient outcomes.

### Example 8: GDM Models Consistently Predict Disease Risk in First Trimester

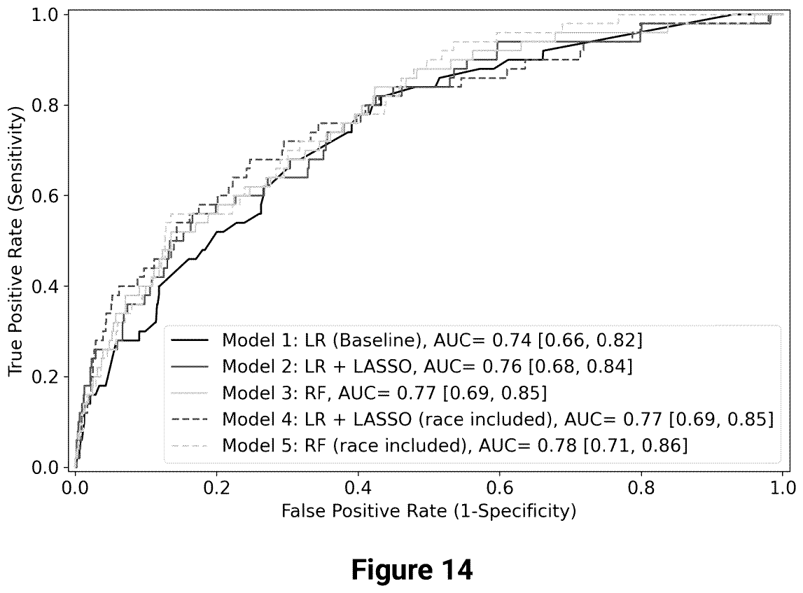

FIG. 14 depicts a comparison of five machine learning models for prediction of GDM in the first trimester with high confidence. The primary outcome is diagnosis with gestational diabetes based on meeting one of the following criteria:

Glucose control test (GCT)>200 mg/dL; OR GCT>130 mg/dL AND two or more of the following on the 3-hour glucose tolerance test (GTT): Fasting>=95 mg/dL, Hour 1>=180 mg/dL, Hour 2>=155 mg/dL, Hour 3>=140 mg/dL; OR one or more of the following on the 2-hour GTT: Fasting>=92 mg/dL, Hour 1>=180 mg/dL, Hour 2>=153 mg/dL. Table 4 below gives information on the employed models:

In the five employed models, the key parameters include ACOG guideline variables (models 1-5): BMI, first-degree relative with diabetes, hypertension, PCOS, cardiovascular disease, race and ethnicity, as well as additional variables in models 2-5: age, gravidity, tobacco use, insurance type, asthma, migraines, blood type, hemoglobin, MCV, hematocrit, platelet PAPP-A, nuchal translucency, anxiety, depression, urine culture, where models 2 and 4 excluded race and ethnicity. Based upon the AUC values obtained, each model performed well to predict GDM, however including lab tests and past diagnostic history (i.e., the additional variables) generally improves model performance. In addition, there is a notable increase in performance when including race and ethnicity in the models. As a whole, these results indicate the robustness of the described models for predicting GDM in the first trimester of pregnancy.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, attributes, or memory contents. Information, arguments, attributes, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-Ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

1-20. (canceled)

21. A computer-implemented method for detecting pregnancy disorders, comprising:
receiving, by a computer from one or more databases, input data for training data associated with a pregnancy disorder outcome, wherein the input data includes respective pluralities of health parameters for respective prior patients having experienced the pregnancy disorder outcome;
selecting, by the computer, respective outcome-relevant sub-pluralities of the respective pluralities of health parameters, wherein the selecting is based upon a known outcome relevance or a computed outcome relevance based upon a machine learning output;
allocating, by the computer, respective outcome-relevant sub-pluralities of parameters for respective patients into training data and testing data;
fitting, by the computer, one or more machine learning models using the training data;
evaluating, by the computer, the performance of the one or more machine learning models using the testing data to select a best-performing machine learning model based upon statistical comparisons of performance among the one or more machine learning models;
outputting, by the computer, one or more variables from the selected best-performing machine learning model, wherein the one or more variables are associated with one or more statistically related input parameters; and
updating, by the computer, a classification threshold for the selected machine learning model as a risk probability cutoff for a given patient developing the pregnancy disorder outcome.

22. The method of claim 21, further comprising determining exclusion conditions for excluding any of the respective pluralities of health parameters based upon a determination of whether any respective prior patients meet the exclusion conditions.

23. The method of claim 21, further comprising determining the classification threshold for the selected machine learning model as a risk probability cutoff for a given patient developing the pregnancy disorder outcome.

24. The method of claim 21, further comprising:
transforming respective associated sub-pluralities having a number of parameters from the respective pluralities of health parameters into a respective transformed parameter having a fewer number of parameters; and
replacing said respective sub-pluralities with said respective transformed parameters.

25. The method of claim 21, further comprising identifying one or more missing parameters in one or more of the respective pluralities of health parameters.

26. The method of claim 25, further comprising identifying at least one missing parameter as informative toward the disease outcome.

27. The method of claim 25, further comprising:
identifying at least one missing parameter as non-informative; and
imputing a representative value to replace the missing parameter.

28. The method of claim 27, wherein the imputation is based upon K-nearest neighbors.

29. The method of claim 21, wherein complex input data having multiple values of respective dependent parameters for respective independent parameters is transformed into a number of finite parameters.

30. The method of claim 21, further comprising, in response to determining that the testing set produces an imbalanced distribution of an outcome variable, balancing the training set by undersampling a majority input parameter or oversampling a minority input parameter.

31. The method of claim 21, further comprising reducing the machine learning model to produce a reduced number of output variables associated with statistically related input parameters.

32. The method of claim 21, further comprising:
identifying one or more pregnancy indicators for a patient based upon one or more types of data stored in one or more data records associated with the patient; and
detecting an instance of a pregnancy in response to determining that the one or more pregnancy indicators satisfy a pregnancy detection threshold.

33. The method of claim 21, wherein the computer receives a selection input indicating the known outcome relevance for an outcome-relevant sub-plurality of a plurality of health parameters from a computing device associated with a clinical expert.

34. A system for detecting pregnancy disorders, comprising:
a computing device comprising at least one processor, configured to:
receive, from one or more databases, input data for training data associated with a pregnancy disorder outcome, wherein the input data includes respective pluralities of health parameters for respective prior patients having experienced the pregnancy disorder outcome;
select respective outcome-relevant sub-pluralities of the respective pluralities of health parameters, wherein selecting is based upon a known outcome relevance or a computed outcome relevance based upon a machine learning output;
allocate respective outcome-relevant sub-pluralities of parameters for respective patients into training data and testing data;
fit one or more machine learning models using the training data;
evaluate the performance of the one or more machine learning models using the testing data to select a best-performing machine learning model based upon statistical comparisons of performance among the one or more machine learning models;
output one or more variables from the selected best-performing machine learning model, wherein the one or more variables are associated with one or more statistically related input parameters; and
update a classification threshold for the selected machine learning model as a risk probability cutoff for a given patient developing the pregnancy disorder outcome.

35. The system of claim 34, wherein the computer is further configured to determine exclusion conditions for excluding any of the respective pluralities of health parameters based upon a determination of whether any respective prior patients meet the exclusion conditions.

36. The system of claim 34, wherein the computer is further configured to determine the classification threshold for the selected machine learning model as a risk probability cutoff for a given patient developing the pregnancy disorder outcome.

37. The system of claim 34, wherein the computer is further configured to:
transform respective associated sub-pluralities having a number of parameters from the respective pluralities of health parameters into a respective transformed parameter having a fewer number of parameters; and
replace said respective sub-pluralities with said respective transformed parameters.

38. The system of claim 34, wherein the computer is further configured to reduce the machine learning model to produce a reduced number of output variables associated with statistically related input parameters.

39. The system according to claim 34, wherein the computer is further configured to:
identify one or more pregnancy indicators for a patient based upon one or more types of data stored in one or more data records associated with the patient; and
detect an instance of a pregnancy in response to determining that the one or more pregnancy indicators satisfy a pregnancy detection threshold.

40. The system according to claim 34, wherein the computer receives a selection input indicating the known outcome relevance for an outcome-relevant sub-plurality of a plurality of health parameters from a computing device associated with a clinical expert.
